(19) United States
(12) Patent Application Publication
(10) Pub. No.: US 2024/0026426 A1
(43) Pub. Date: Jan. 25, 2024

(54) DECOY OLIGONUCLEOTIDES AND RELATED METHODS

(71) Applicant: 10x Genomics, Inc., Pleasanton, CA (US)

(72) Inventor: Felice Alessio BAVA, Rome (IT)

(21) Appl. No.: 18/356,933

(22) Filed: Jul. 21, 2023

Related U.S. Application Data
(60) Provisional application No. 63/391,564, filed on Jul. 22, 2022.

(51) Int. Cl. C12Q 1/6837; C12Q 1/6844

(52) U.S. Cl. CPC C12Q 1/6837; C12Q 1/6844; C12Q 2600/166

(57) ABSTRACT

In some aspects, the present disclosure relates to methods for reducing the detection of false positive events during analysis of a biological sample. In some aspects, the method comprises use of a decoy oligonucleotide that can hybridize to a probe or to a target nucleic acid. The methods herein have particular applicability in reducing the detection of false positive events and in combining hybridization and ligation reactions into a single step. Also provided are kits comprising probes for use in such methods.

### CROSS-REFERENCE TO RELATED APPLICATIONS

This application claims priority to U.S. Provisional Patent Application No. 63/391,564, filed Jul. 22, 2022, entitled “DECOY OLIGONUCLEOTIDES AND RELATED METHODS,” which is herein incorporated by reference in its entirety for all purposes.

### FIELD

The present disclosure generally relates to methods and compositions for in situ analysis or detection of analytes in a sample.

### BACKGROUND

Methods are available for analyzing nucleic acids in a biological sample in situ, such as a cell or a tissue. For instance, advances in single molecule fluorescent hybridization (smFISH) have enabled nanoscale-resolution imaging of RNA in cells and tissues. However, oligonucleotide probe-based assay methods for in situ analysis may suffer from low sensitivity, specificity, and/or detection efficiency and may require careful and laborious optimization. Improved methods for in situ analysis are needed. The present disclosure addresses these and other needs.

### BRIEF SUMMARY

In some aspects, provided herein is a method for analyzing a biological sample, comprising: a) contacting the biological sample, a probe or probe set, and a decoy oligonucleotide with one another in any suitable order, wherein the biological sample comprises a target nucleic acid comprising a target region, the probe or probe set comprises a hybridization region, and the decoy oligonucleotide comprises a decoy region capable of hybridizing to the hybridization region or the target region; b) allowing the probe or probe set and the target nucleic acid to hybridize at one or more locations in the biological sample; and c) detecting a signal associated with the probe or probe set or a product thereof at the one or more locations, thereby detecting the target nucleic acid in the biological sample. In some embodiments, the decoy oligonucleotide reduces hybridization between the hybridization region and an off-target region in the biological sample. For example, the decoy oligonucleotide reduces hybridization between the hybridization region and an off-target region in the biological sample compared to in the absence of the decoy oligonucleotide.

In some embodiments, the decoy region has less than 98% sequence identity to the hybridization region of the probe or probe set. In any of the preceding embodiments, the decoy region can have a lower sequence complementarity to the target region compared to the sequence complementarity of the hybridization region to the target region. In any of the preceding embodiments, the decoy region may have less than 98% sequence complementarity to the target region. In any of the preceding embodiments, the decoy region may have between about 80% and about 95% sequence complementarity to the target region. In any of the preceding embodiments, the hybridization region may have a higher sequence complementarity to the target region compared to the sequence complementarity of the hybridization region to the decoy region. In any of the preceding embodiments, the hybridization region can have at least 95% sequence complementarity to the decoy region. In any of the preceding embodiments, the hybridization region can have at least 99% sequence complementarity to the decoy region.

In some embodiments, the hybridization region has a higher sequence complementarity to the target region than to the off-target region. In some embodiments, the hybridization region has at least 95% sequence complementarity to the target region and less than 95% sequence complementarity to the off-target region. In some embodiments, the hybridization region has at least 99% sequence complementarity to the target region and between about 80% and about 95% sequence complementarity to the off-target region.

In any of the preceding embodiments, the decoy region can have a higher sequence complementarity to the off-target region than to the target region. In any of the preceding embodiments, the decoy region can have at least 95% sequence complementarity to the off-target region and less than 95% sequence complementarity to the target region. In any of the preceding embodiments, the decoy region can have at least 99% sequence complementarity to the off-target region and between about 80% and about 95% sequence complementarity to the target region.

In some embodiments, the hybridization region has a higher sequence complementarity to the decoy region than to the off-target region. In some embodiments, the hybridization region has at least 95% sequence complementarity to the decoy region and less than 95% sequence complementarity to the off-target region. In some embodiments, the hybridization region has at least 99% sequence complementarity to the decoy region and between about 80% and about 95% sequence complementarity to the off-target region.

In any of the preceding embodiments, the decoy oligonucleotide can be detectably labeled or not detectably labeled. In some embodiments, the decoy oligonucleotide is not detectably labeled. In some embodiments, upon hybridization to the off-target region or the hybridization region, the decoy oligonucleotide does not comprise a region capable of directly or indirectly binding to a detectably labeled probe. In any of the preceding embodiments, upon hybridization to the off-target region or the hybridization region, the decoy oligonucleotide may not be ligatable with itself, within the probe set, or with another oligonucleotide. In any of the preceding embodiments, upon hybridization to the off-target region or the hybridization region, the decoy oligonucleotide may not be detectable by detectable probes configured to detect the probe or probe set or product thereof. In any of the preceding embodiments, upon hybridization to the off-target region or the hybridization region, the decoy oligonucleotide may not be capable of generating a product that is detectable by detectable probes configured to detect the probe or probe set or product thereof. In any of the preceding embodiments, the product can be a rolling circle amplification (RCA) product.

In any of the preceding embodiments, the probe or probe set can be selected from the group consisting of: a probe comprising a 3′ or 5′ overhang upon hybridization to the target nucleic acid. In some embodiments, the 3′ or 5′ overhang comprises one or more detectable labels and/or barcode sequences; a probe comprising a 3′ overhang and a 5′ overhang upon hybridization to the target nucleic acid. In some embodiments, the 3′ overhang and the 5′ overhang each independently comprises one or more detectable labels and/or barcode sequences; a circular probe; a circularizable probe or probe set; a probe or probe set comprising a split hybridization region configured to hybridize to a splint. In some embodiments, the split hybridization region comprises one or more barcode sequences; and a combination thereof. In any of the preceding embodiments, the probe or probe set can be detectably labeled. In any of the preceding embodiments, the probe or probe set is not detectably labeled. In any of the preceding embodiments, the probe or probe set can further comprise a region capable of directly or indirectly binding to a detectably labeled probe. In any of the preceding embodiments, upon hybridization to the target region, the probe or probe set can be ligatable with itself, within the probe set, or with another oligonucleotide.

In any of the preceding embodiments, the probe or probe set can be ligatable using the target region as template, with or without flap cleavage and with or without gap filling prior to ligation. In any of the preceding embodiments, upon hybridization to the target region, the probe or probe set can be capable of generating a product. In any of the preceding embodiments, the product of the probe or probe set can be a rolling circle amplification (RCA) product generated in situ at a location in the biological sample. In any of the preceding embodiments, the method can comprise prior to the detecting, a step of removing a complex comprising the probe or probe set hybridized to the decoy oligonucleotide from the biological sample.

In some aspects, provided herein is a method for analyzing a biological sample, comprising: a) contacting the biological sample, a circularizable probe or probe set, and a decoy oligonucleotide with one another in any suitable order, wherein: the biological sample comprises a target nucleic acid comprising a target region, the circularizable probe or probe set comprises a first hybridization region and a second hybridization region which, upon hybridization to the target region, are ligatable, and the decoy oligonucleotide comprises a decoy region capable of hybridizing to the first and/or second hybridization regions; b) allowing the circularizable probe or probe set and the target nucleic acid to hybridize at one or more locations in the biological sample, wherein the decoy oligonucleotide reduces hybridization between the first and/or second hybridization regions and an off-target region in the biological sample; c) circularizing the circularizable probe or probe set to generate a circular probe by ligating the first and second hybridization regions using the target region as template, with or without flap cleavage and with or without gap filling prior to ligation; d) generating a rolling circle amplification (RCA) product of the circular probe; and e) detecting a signal associated with the RCA product at the one or more locations, thereby detecting the target nucleic acid in the biological sample.

In any of the preceding embodiments, the circularizable probe or probe set can be pre-hybridized to the decoy oligonucleotide. In any of the preceding embodiments, the target region can displace the decoy region hybridized to the circularizable probe or probe set, thereby hybridizing the circularizable probe or probe set to the target nucleic acid. In any of the preceding embodiments, the hybridization of the probe or probe set and the target nucleic acid and the ligation of the probe or probe set can be carried out under the same reaction condition. In some instances, a ligase that performs the ligation can be added prior to, during, and/or after the hybridization of the probe or probe set and the target nucleic acid. In any of the preceding embodiments, the ligase can be present in and/or added to a reaction buffer for the hybridization of the probe or probe set and the target nucleic acid. In any of the preceding embodiments, the method can avoid washing the biological sample and/or changing a reaction buffer between the hybridization of the probe or probe set and the target nucleic acid and the ligation of the probe or probe set. In any of the preceding embodiments, the method can avoid washing the biological sample and/or changing a reaction buffer between the contacting of the biological sample with a probe or probe set and a decoy oligonucleotide and the ligation of the probe or probe set.

In any of the preceding embodiments, the method can comprise, prior to the circularizing of the probe or probe set, a step of removing a complex comprising the circularizable probe or probe set hybridized to the decoy oligonucleotide from the biological sample. However, in some embodiments, a complex comprising the circularizable probe or probe set hybridized to the decoy oligonucleotide is not removed from the biological sample prior to the circularizing of the probe or probe set.

In any of the preceding embodiments, the decoy oligonucleotide can comprise one or more mismatches with the circularizable probe or probe set at or near a ligation junction. In any of the preceding embodiments, a circular probe of the circularizable probe or probe set hybridized to the decoy oligonucleotide may not be generated. In any of the preceding embodiments, the decoy oligonucleotide may not be capable of being extended by a polymerase. In some embodiments, the decoy oligonucleotide comprises an irreversible terminating group. In some embodiments, the decoy oligonucleotide can comprise a 3′ dideoxynucleotide.

In some aspects, provided herein is a method for analyzing a biological sample, comprising: a) contacting the biological sample, a circularizable probe or probe set, and a decoy oligonucleotide with one another in any suitable order, wherein: the biological sample comprises a target nucleic acid comprising a target region, the circularizable probe or probe set comprises a first hybridization region and a second hybridization region which, upon hybridization to the target region, are ligatable, and the decoy oligonucleotide comprises a decoy region capable of hybridizing to an off-target region; b) allowing the circularizable probe or probe set and the target nucleic acid to hybridize at one or more locations in the biological sample, wherein the decoy oligonucleotide reduces hybridization between the first and/or second hybridization regions and the off-target region in the biological sample; c) circularizing the circularizable probe or probe set to generate a circular probe by ligating the first and second hybridization regions using the target region as template, with or without flap cleavage and with or without gap filling prior to ligation; d) generating a rolling circle amplification (RCA) product of the circular probe; and e) detecting a signal associated with the RCA product at the one or more locations, thereby detecting the target nucleic acid in the biological sample. In some embodiments, the decoy region is capable of hybridizing to the target region. In some embodiments, the decoy region hybridizes to the off-target region with a higher affinity than to the target region.

In any of the preceding embodiments, the off-target region can be pre-hybridized to the decoy oligonucleotide. In any of the preceding embodiments, the target region and the off-target region can be pre-hybridized to the decoy oligonucleotide. In any of the preceding embodiments, the first and/or hybridization regions can displace the decoy region hybridized to the target region, thereby hybridizing the circularizable probe or probe set to the target nucleic acid. In any of the preceding embodiments, the first and/or hybridization regions may not displace the decoy region hybridized to the off-target region.

In any of the preceding embodiments, the hybridization of the probe or probe set and the target nucleic acid and the ligation of the probe or probe set can be carried out under the same reaction condition. In any of the preceding embodiments, a ligase that performs the ligation can be added prior to, during, and/or after the hybridization of the probe or probe set and the target nucleic acid. In any of the preceding embodiments, the ligase can be present in and/or added to a reaction buffer for the hybridization of the probe or probe set and the target nucleic acid. In any of the preceding embodiments, the method can avoid washing the biological sample and/or changing a reaction buffer between the hybridization of the probe or probe set and the target nucleic acid and the ligation of the probe or probe set. In any of the preceding embodiments, the method can avoid washing the biological sample and/or changing a reaction buffer between the contacting of the biological sample with a probe or probe set and a decoy oligonucleotide and the ligation of the probe or probe set.

In any of the preceding embodiments, a first decoy oligonucleotide hybridized to the target region can be removed from the biological sample prior to the circularizing of the probe or probe set. In any of the preceding embodiments, a second decoy oligonucleotide hybridized to the off-target region may not be removed from the biological sample prior to the circularizing of the probe or probe set. In any of the preceding embodiments, the decoy oligonucleotide can circularizable, wherein in a first complex the decoy oligonucleotide comprises one or more mismatches with the target region at or near a ligation junction. In any of the preceding embodiments, in the second complex the decoy oligonucleotide may not comprise a mismatch with the off-target region at or near a ligation junction.

In any of the preceding embodiments, in the first complex and/or the second complex, the decoy oligonucleotide can comprise a non-ligatable 3′ end and/or non-ligatable 5′ end. In any of the preceding embodiments, in the circularizing of the probe or probe set, a circular probe is not generated from the decoy oligonucleotide hybridized to the target region or the off-target region. In any of the preceding embodiments, the decoy oligonucleotide may lack a phosphate group at the 5′ end. In any of the preceding embodiments, the decoy oligonucleotide can comprise one or more modifications that reduce its ability to be used as a template for amplification. In any of the preceding embodiments, the decoy oligonucleotide can comprise one or more modifications that facilitate removal of the first complex as compared to removal of the second complex.

In any of the preceding embodiments, complementarity between the decoy region of the decoy oligonucleotide and the hybridization region in the probe or probe set can be lower than complementarity between the hybridization region and a target nucleic acid. In any of the preceding embodiments, the decoy oligonucleotide has between 80% and about 95% complementarity to the hybridization region in the probe or probe set.

In any of the preceding embodiments, probe or probe set and the decoy oligonucleotide can be provided as the first complex. Alternatively, in any of the preceding embodiments, the probe or probe set and the decoy oligonucleotide can be provided separately. In some embodiments, the method can further comprise allowing hybridization of the probe or probe set and the decoy oligonucleotide to form the first complex. In any of the preceding embodiments, the probe or probe set and the decoy oligonucleotide can be provided at a ratio of 1:1. In any of the preceding embodiments, the probe or probe set and the decoy oligonucleotide can be provided at a ratio higher than 1:1.

In some embodiments, the decoy oligonucleotide may be no more than about 10, no more than about 15, no more than about 20, no more than about 25, no more than about 30, no more than about 35, no more than about 40, no more than about 45, no more than about 50, no more than about 60, no more than about 70, no more than about 80, no more than about 90, or no more than about 100 nucleotides in length. In any of the preceding embodiments, the decoy oligonucleotide may be no more than 10, no more than 15, no more than 20, no more than 25, or no more than 30 nucleotides in length.

In any of the preceding embodiments, the method can further comprise removing the decoy probe hybridized to the first and/or second hybridization region or the target region prior to ligating the probe or probe set hybridized to the target nucleic acid. In some embodiments, the removing step can comprise one or more stringency washes.

In any of the preceding embodiments, the target nucleic acid can be RNA or DNA. In some embodiments, the target nucleic acid is an mRNA. In some embodiments, the target nucleic acid is a noncoding RNA. In any of the preceding embodiments, the target region can comprise a single nucleotide of interest, an alternatively spliced region, a deletion, and/or a frameshift. In some embodiments, the single nucleotide of interest is selected from the group consisting of a single-nucleotide polymorphism (SNP), a single-nucleotide variant (SNV), a single-nucleotide substitution, a point mutation, or a single-nucleotide insertion. In some embodiments, the single nucleotide of interest is a SNP. In some embodiments, the single nucleotide of interest is a point mutation.

In any of the preceding embodiments, the target nucleic acid can be in a tissue sample. In any of the preceding embodiments, the target region can be analyzed in situ (e.g., at a location) in the tissue sample or in a matrix embedding the tissue sample. In any of the preceding embodiments, the tissue sample can be an intact tissue sample or a non-homogenized tissue sample. In any of the preceding embodiments, the target nucleic acid can be in a cell in the tissue sample. In any of the preceding embodiments, the method can comprise permeabilizing the cell before, during, or after the contacting step. In any of the preceding embodiments, the tissue sample can be a tissue section. In any of the preceding embodiments, the tissue sample can a fixed tissue sample, a frozen tissue sample, or a fresh tissue sample. In some embodiments, the tissue sample can be a formalin-fixed, paraffin-embedded (FFPE) sample. In any of the preceding embodiments, the probe or probe set, the ligated probe or probe set, and/or the amplification product thereof can be immobilized in the sample and/or crosslinked to one or more other molecules in the sample.

In any of the preceding embodiments, the ligating can be enzymatic ligation or chemical ligation. In any of the preceding embodiments, the ligating can be performed using a ligase selected from the group consisting of a T4 RNA ligase 1, a T4 RNA ligase 2 or a PBCV-1 DNA ligase. In any of the preceding embodiments, ligating the probe or probe set may result in a circularized probe. In any of the preceding embodiments, detecting the ligated probe or probe set can comprise generating an amplification product in situ, and detecting the amplification product. In any of the preceding embodiments, detecting the amplification product can comprise determining a sequence of the amplification product. In some instances, detecting the amplification product can comprise sequencing all or a portion of the amplification product. In some embodiments, the sequencing can comprise sequencing by hybridization, sequencing by ligation, and/or fluorescent in situ sequencing. In some instances, detecting the amplification product can comprise in situ hybridization to the amplification product. In some instances, the in situ hybridization can comprise sequential fluorescent in situ hybridization. In some embodiments, a sequence in the amplification product indicative of the target region can be determined. In any of the preceding embodiments, detecting the probe or probe set, the ligated probe or probe set, and/or the amplification product can comprise labeling the probe or probe set, the ligated probe or probe set, and/or the amplification product with a fluorophore, an isotope, a mass tag, or a combination thereof.

In any of the preceding embodiments, the amplification product can be generated using a linear rolling circle amplification (RCA), a branched RCA, a dendritic RCA, or any combination thereof. In any of the preceding embodiments, the amplification product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any the preceding embodiments, the method can comprise imaging the sample to detect the probe or probe set, the ligated probe or probe set, and/or the amplification product thereof. In any the preceding embodiments, the imaging can comprises detecting a signal associated the probe or probe set, the ligated probe or probe set, and/or the amplification product thereof. In any the preceding embodiments, the signal can be amplified in situ in the sample. In some embodiments, the signal amplification in situ can comprise RCA of a probe that directly or indirectly binds to the probe or probe set and/or the amplification product thereof; hybridization chain reaction (HCR) directly or indirectly on the probe or probe set and/or the amplification product thereof; linear oligonucleotide hybridization chain reaction (LO-HCR) directly or indirectly on the probe or probe set and/or the amplification product thereof; primer exchange reaction (PER) directly or indirectly on the probe or probe set and/or the amplification product thereof, assembly of branched structures directly or indirectly on the probe or probe set and/or the amplification product thereof; hybridization of a plurality of detectable probes directly or indirectly on the probe or probe set and/or the amplification product thereof, or any combination thereof.

In any of the preceding embodiments, the probe or probe set can comprise one or more barcode sequences. In any of the preceding embodiments, the probe or probe set can comprise one or more barcode sequences that identifies a nucleic acid sequence. In some embodiments, the one or more barcode sequences can identify the target region. In any of the preceding embodiments, the one or more barcode sequences can be between about 8 and about 16 nucleotides in length. In any the preceding embodiments, the one or more barcode sequences can be between about 8 and about 10 nucleotides in length.

In any the preceding embodiments, the method can comprise detecting the one or more barcode sequences by: contacting the biological sample with one or more detectably-labeled probes that directly or indirectly hybridize to the one or more barcode sequences, detecting signals associated with the one or more detectably-labeled probes, and dehybridizing the one or more detectably-labeled probes. In any of the preceding embodiments, the contacting, detecting, and dehybridizing steps can be repeated with the one or more detectably-labeled probes and/or one or more other detectably-labeled probes that directly or indirectly hybridize to the one or more barcode sequences.

In some aspects, provided herein is a method for analyzing a biological sample, comprising: a) contacting the biological sample, with a complex comprising a circularizable probe and a decoy oligonucleotide, wherein: the biological sample comprises a target nucleic acid comprising a target region, the circularizable probe comprises a first hybridization region and a second hybridization region which, upon hybridization to the target region, are ligatable, and the decoy oligonucleotide comprises a decoy region capable of hybridizing to the first and/or second hybridization regions, wherein the complementarity between the decoy region and the first and/or second hybridization region is lower than the complementarity between the target region and the first and/or second hybridization region, but the complementarity between the decoy region and the first and/or second hybridization region is higher than the complementarity between an off-target region and the first and/or second hybridization region; b) allowing the circularizable probe to hybridize to the target nucleic acid at one or more locations in the biological sample, thereby displacing the decoy oligonucleotide; c) circularizing the circularizable probe to generate a circular probe by ligating the first and second hybridization regions using the target region as template, wherein the ligating is performed under the same reaction conditions as the hybridizing in step b); d) generating a rolling circle amplification (RCA) product of the circular probe; and e) detecting a signal associated with the RCA product at the one or more locations, thereby detecting the target nucleic acid in the biological sample.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 depicts an exemplary probe comprising a hybridization region complementary to a target region in a target nucleic acid, and a decoy target comprising a decoy region that hybridizes to the hybridization region in the probe, preventing or reducing its hybridization to an off-target region in an off-target nucleic acid.

FIG. 2 depicts an exemplary probe comprising a hybridization region complementary to a target region in a target nucleic acid, and a decoy probe comprising a decoy region that binds an off-target region in an off-target nucleic acid.

FIGS. 3A-3C depict various exemplary decoy target designs.

FIGS. 4A-4D depict various exemplary decoy probe designs.

FIG. 5 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ methods for detecting sequences of interest in target nucleic acids have typically been performed using fluorescent in situ hybridization (FISH). However, background from off-target binding of FISH probes can become limiting in a number of important applications, such as increasing the degree of multiplexing, imaging shorter RNAs, detecting short specific sequences of interest (such as SNPs) and imaging tissue samples. Approaches including templated ligation may increase specificity by requiring ligation of a probe or probe set in order to detect a ligated probe or probe set or an amplification or extension product thereof (such as a rolling circle amplification product). However, such approaches typically require separate steps for hybridization and multiple wash steps prior to ligation to ensure that only specifically hybridized probes are ligated. Thus, there is a need for more efficient and specific methods for detection of target nucleic acids in situ. The present application addresses these and other needs.

In some aspects, provided herein are decoy oligonucleotides and methods of using said decoy oligonucleotides to reduce off-target hybridization of a probe or probe set in a sample. Fluorescent in situ hybridization (FISH) techniques are becoming extremely sensitive, to the point where individual RNA or DNA molecules can be detected with small probes. At this level of sensitivity, the elimination of off-target hybridization is of crucial importance, but typical probes used for RNA and DNA FISH contain sequences repeated elsewhere in the genome. In some instances, detection of signals associated with a probe or probe set is performed in situ at one or more locations in the sample and elimination of a false positive signal (e.g., from off-target hybridization) is important for accurate analyte detection. In some embodiments, the products associated with the probe or probe set are not eluted from the sample for collection, capture and/or is not further processed before detection. In some embodiments, elimination of a false positive signal (e.g., from off-target hybridization) is important for accurate analyte detection in a complex environment such as a tissue sample. In some embodiments, the decoy oligonucleotides comprise a decoy region that hybridizes to a probe or probe set and thereby reduces hybridization of the probe or probe set to an off-target region (e.g., a region in an off-target nucleic acid molecule). In some embodiments, the decoy region of the decoy oligonucleotide has higher sequence complementarity to the hybridization region of the probe or probe set than the off-target region has to the hybridization region of the probe or probe set. In some embodiments, the decoy oligonucleotides comprise a decoy region that hybridizes to an off-target region and thereby reduces hybridization of the probe or probe set to an off-target region. In some embodiments, the decoy oligonucleotide hybridizes to the target region. In some embodiments, the probe or probe set hybridizes to the target nucleic acid in the presence of the decoy oligonucleotide, but does not hybridize to an off-target region in the presence of the decoy oligonucleotide or hybridizes at a lower rate to an off-target region in the presence of the decoy oligonucleotide.

In some embodiments, the probe or probe set is ligatable. In some embodiments, the probe or probe set is designed such that ligation of the probe or probe set can be templated by the target region upon specific hybridization to the target region. In some embodiments, the probe or probe set is a circularizable probe or probe set. In some embodiments, the probe or probe set comprises a first and second probe that can be ligated together upon hybridization to the target region to form a ligated first-second probe. In some embodiments, the probe or probe set is ligatable, and the method comprises contacting the sample with a decoy oligonucleotide (e.g., a decoy target) that hybridizes to the probe or probe set but does not serve as a template for ligation of the probe or probe set. In some embodiments, the decoy oligonucleotide does not bridge a split hybridization region in the probe or probe set (e.g., the decoy oligonucleotide hybridizes only to one probe or to one end of a circularizable probe or probe set, and cannot serve as a template for ligation). In some embodiments, the decoy oligonucleotide comprises one or more point mutations at or near the splice junction that prevent ligation of the probe or probe set using the decoy oligonucleotide as a template. In some embodiments, the decoy oligonucleotide can additionally or alternatively include one or more moieties or modifications that prevent its extension by a polymerase. In some embodiments, the decoy oligonucleotide can additionally or alternatively include one or more modifications that facilitate removal of a complex comprising the probe or probe set hybridized to the decoy oligonucleotide from the sample. Decoy oligonucleotides and probes or probe sets according to the present application are described in further detail in Section II.

The present disclosure provides methods and compositions for analysis of target regions in target nucleic acids. In some embodiments, the probe or probe set can distinguish between a single nucleotide variation in a target region and off-target region in the presence of a decoy oligonucleotide. In some embodiments, the target region is a region comprising a single nucleotide of interest, and the off-target region comprises a different single nucleotide variation (e.g., a SNP or other point mutation, or a single nucleotide insertion or deletion). In some embodiments, the target region comprises a single nucleotide of interest (e.g., SNPs or point mutations), a dinucleotide sequence, or a short sequence of about 5 nucleotides in length, or longer sequences.

In some aspects, a target nucleic acid disclosed herein comprises any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). The target may, in some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present application allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

Provided herein are methods involving the use of a probe or probe set and a decoy oligonucleotide for analyzing one or more target nucleic acid(s), such as a target nucleic acid (for example, a messenger RNA) present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof (e.g., presence or absence of sequence variants such as point mutations and SNPs). In some aspects, the provided methods can be applied to detect, image, quantitate, or determine the sequence of one or more target nucleic acid(s), comprising sequence variants such as point mutations and SNPs. In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, the provided methods involve a step of contacting, or hybridizing, one or more polynucleotides, such as any of the probes described herein, to a target nucleic acid with a target region in order to form a hybridization complex. In some aspects, the provided methods comprise one or more steps of ligating the polynucleotides, for instance of ligating the ends of a circularizable probe to form a circularized probe, or ligating a first probe and a second probe to form a ligated first-second probe (which may be a linear probe). In some aspects, the provided methods involve a step of amplifying one of the polynucleotides (e.g., a padlock probe or a circularized probe produced therefrom), to generate an amplification product. Various aspects of hybridization, ligation, extension, and/or amplification steps are described in Section III below.

In some aspects, the provided methods involve detecting the probe or probe set or a product thereof in situ in the biological sample (e.g., at a spatially localized position in the biological sample). In some aspects, the provided methods involve a step of detecting and/or determining the sequence of all or a portion of a probe or probe set or a product thereof (e.g., a ligation, extension, and/or amplification product thereof), such as one or more barcode sequences in the probe or probe set or product thereof. In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Aspects of detecting probes or probe sets, or products thereof, are described in Section IV below.

In some aspects, the present application provides compositions comprising the decoy oligonucleotides described herein and kits for use according to the methods described herein, as described in Section V below.

In some embodiments, a provided method is quantitative and preserves the spatial information within a tissue sample without physically isolating cells or using homogenates. In some embodiments, the provided methods for analyzing a biological sample allow detection of one or more analytes (e.g., any nucleic acid or protein analytes) in the biological sample. Aspects of samples and analytes that can be analyzed according using the provided methods, compositions, and kits are described in Section VII below.

### II. Polynucleotides and Hybridization Complexes

Disclosed herein in some aspects are nucleic acid probes and/or probe sets that are introduced into a cell or used to otherwise contact a biological sample such as a tissue sample. The probes may comprise any of a variety of entities that can hybridize to a nucleic acid, typically by Watson-Crick base pairing, such as DNA, RNA, LNA, PNA, etc., depending on the application. The nucleic acid probe(s) typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease for cleavage).

In some aspects, the nucleic acid probes and/or probe sets comprise a hybridization region, wherein the hybridization region on the probe is capable of hybridizing to a target region on the target nucleic acid or to a decoy region in a decoy oligonucleotide. In some aspects, the detection specificity and stringency using the probe or probe set is increased by hybridization of the probe or probe set, or of a target nucleic acid, to a decoy oligonucleotide which reduces hybridization of the probe or probe set to an off-target nucleic acid (or an off-target region of a nucleic acid) and promotes hybridization of the probe or probe set to the target nucleic acid. In some embodiments, the decoy region in the decoy oligonucleotide is hybridized to the hybridization region in the probe or probe set. In some embodiments, the decoy region in the decoy oligonucleotide is hybridized to the target region in the target nucleic acid.

In some embodiments, the methods provided herein comprise contacting a biological sample, with a probe or probe set, wherein the biological sample comprises a target nucleic acid comprising a target region and the probe or probe set comprises a hybridization region capable of hybridizing to the target region. In some embodiments, the hybridization region is a split hybridization region (e.g., a hybridization region comprising at least a first portion and a second portion that are separated by the absence of a linkage, optionally wherein the first portion and the second portion of the hybridization region are separated by a gap of 1, 2, 3, 4, 5, or more nucleotides when hybridized to the target region). In some embodiments, the method comprises ligating the split hybridization region to generate a ligated probe using the target region as a template. In some embodiments, the hybridization region of the probe or probe set has a higher sequence complementarity to the target region than to the off-target region. In some embodiments, the hybridization region has at least 95% sequence complementarity to the target region and less than 95% sequence complementarity to the off-target region. In some embodiments, the hybridization region has at least 99% sequence complementarity to the target region and between about 80% and about 95% sequence complementarity to the off-target region. In some embodiments, the hybridization region has 100% sequence complementarity to the target region and less than 100% complementarity to the off-target region. In some embodiments, the hybridization region has 100% complementarity to the target region and between about 80% and about 95% complementarity to the off-target region.

In some embodiments, an off-target region is a sequence present in a nucleic acid in a biological sample that has significant homology to a target region in the biological sample. In some embodiments, the target region is present in a target nucleic acid (e.g., an mRNA molecule of interest or corresponding cDNA) and the off-target region is present in an off-target nucleic acid (e.g., a different mRNA molecule or corresponding cDNA). In some embodiments, the target region is present in an mRNA molecule of a first gene and the off-target region is present in an mRNA molecule of a different gene. In some embodiments, the off-target region comprises a k-mer that is present in the target region. For example, the target region may be a region that is at least 20, 25, 30, 35, 40, or 50 nucleotides in length, wherein the target region is specific to a particular target nucleic acid but a shorter k-mer in the target region (e.g., a 10-mer, 11-mer, 12-mer, 13-mer, 14-mer, 15-mer, 16-mer, 17-mer, or 18-mer in the target region) is present in multiple copies in a set of nucleic acids present in the sample (e.g., the k-mer may be present in multiple copies in the transcriptome, such as in one or more off-target mRNAs in addition to a target mRNA). In some instances, the k-mer is common to multiple different nucleic acids (e.g., multiple different nucleic acid analytes in the sample).

Although various probe design pipelines can be used to minimize the likelihood of off-target probe hybridization, in some cases the presence of repeated k-mers or other off-target regions with high sequence homology to the target region makes it difficult to design specific probes for a given target nucleic acid. In some cases, the quaternary structure of endogenous RNAs can limit the accessibility of some candidate target regions, further limiting the selection of target regions that do not result in significant off-target probe hybridization. In some embodiments, the present application provides decoy and oligonucleotides and methods to avoid or reduce off-target hybridization. In some aspects, the decoy oligonucleotides facilitate use of target regions in target nucleic acids such as mRNA that might otherwise result in significant off-target probe hybridization.

In some embodiments, the decoy oligonucleotide hybridizes to the k-mer that is present in both the target region and the off-target region (in this case, the decoy oligonucleotide can be referred to as a decoy probe). In some embodiments, when the probe or probe set hybridizes to the target region, it is able to displace the decoy oligonucleotide from the target region, but does not displace the decoy oligonucleotide bound to an off-target region. In some embodiments, the probe or probe set out-competes the decoy oligonucleotide for hybridization to the target region, but does not out-compete the decoy oligonucleotide for hybridization to an off-target region. In other embodiments, the decoy oligonucleotide hybridizes to the complement of the particular repeated k-mer in the hybridization region of the probe or probe set (in this case, the decoy oligonucleotide can be referred to as a decoy target). In some embodiments, when the probe or probe set hybridizes to the target region, the target region is able to displace the decoy oligonucleotide from the probe or probe set, but the off-target region does not displace the decoy oligonucleotide from the probe or probe set. In some embodiments, the target region out-competes the decoy oligonucleotide for hybridization to the probe or probe set, but the off-target region does not out-compete the decoy oligonucleotide for hybridization to the probe or probe set.

In some embodiments, the off-target region comprises a k-mer that is identical to a k-mer in the target region. In some embodiments, the off-target region comprises a 5-mer, 6-mer, 7-mer, 8-mer, 9-mer, 10-mer, 15-mer, 16-mer, 17-mer, 18-mer, 19-mer, 20-mer, 25-mer, 30-mer, 35-mer or longer k-mer that is identical to a sequence in the target region. In some embodiments, the off-target region has at least 70%, at least 75%, at least 80%, at least 81%, at least 82%, at least 83%, at least 84%, at least 85%, at least 90%, at least 95%, at least 96%, at least 98%, or at least 99% sequence identity to the target region. In some embodiments, the off-target region is a sequence of the same length as the target region that has at least 70%, at least 75%, at least 80%, at least 81%, at least 82%, at least 83%, at least 84%, at least 85%, at least 90%, at least 95%, at least 96%, at least 98%, or at least 99% sequence identity to the target region.

In some embodiments, an off-target region is identified for a given target region by identifying sequences present in the biological sample that meet a sequence homology threshold for the target region (e.g., at least 70%, at least 75%, at least 80%, at least 81%, at least 82%, at least 83%, at least 84%, at least 85%, at least 90%, at least 95%, at least 96%, at least 98%, or at least 99% sequence identity to the target region). Sequence homology can be determined using any suitable alignment tool, such as the NCBI Basic Local Alignment Search Tool (BLAST). In some embodiments, an alignment tool is used to query a given target region against a database of nucleic acid sequences present in the biological sample. In some embodiments, the database is a species-specific genome and transcriptome database, such as a human, mouse, rat, or other mammalian genome and transcriptome database. In some embodiments, the database is a tissue-specific transcriptome database. Thus, in some embodiments, decoy oligonucleotides can be provided based on the off-target sequences that are present or are likely to be present in a particular biological sample of interest (such as a biological sample from a particular species and/or a particular cell-type or tissue-type).

In some embodiments, a biological sample comprises a plurality of off-target regions corresponding to a given target region. For example, multiple off-target nucleic acid molecules such as off-target mRNAs may comprise off-target regions. The off-target regions can be the same or different. In some instances, at least a first off-target region and a second off-target region having different sequences are present in the biological sample. Each of the multiple off-target regions can be a sequence having at least 70%, at least 75%, at least 80%, at least 81%, at least 82%, at least 83%, at least 84%, at least 85%, at least 90%, at least 95%, at least 96%, at least 98%, or at least 99% sequence identity to the target region. In some instances, each of the off-target regions can be a sequence having at most 95%, at most 90%, at most 85%, at most 80%, at most 75%, at most 70%, at most 65%, at most 60%, sequence identity to the target region. In some embodiments, the same decoy oligonucleotide reduces hybridization of the probe or probe set to the plurality of off-target regions. In some embodiments, a plurality of decoy oligonucleotides are provided for the plurality of off-target regions. For example, different decoy oligonucleotides may be designed that are complementary to the different off-target regions.

Section II.A below describes various decoy oligonucleotide designs in greater detail. The decoy oligonucleotides provided herein can reduce off-target hybridization, off-target detection, and/or off-target product generation (e.g., ligation and/or amplification of probes hybridized to off-target regions) for any type of probe or probe set. Exemplary probes and probe sets include but are not limited to those described in Section II.B below.

### A. Decoy Oligonucleotides

In some aspects, disclosed herein is a method for analyzing a biological sample, comprising contacting the biological sample with a probe or probe set and a decoy oligonucleotide in any suitable order, wherein the biological sample comprises a target nucleic acid comprising a target region, the probe or probe set comprises a hybridization region, and the decoy oligonucleotide comprises a decoy region capable of hybridizing to the hybridization region or the target region, and allowing the probe or probe set and the target nucleic acid to hybridize at one or more locations in the biological sample, wherein the decoy oligonucleotide reduces hybridization between the hybridization region and an off-target region in the biological sample. In some embodiments, the method comprises detecting the probe or probe set, or a product thereof, at one or more locations in the sample. In some embodiments, the decoy oligonucleotide reduces detection of the probe or probe set or product thereof associated with one or more off-target regions in the sample. For example, the decoy oligonucleotide may reduce hybridization of the probe or probe set to one or more off-target regions, and/or may reduce the likelihood that a product is generated from a probe or probe set at one or more off-target regions in the biological sample. The product can be a ligation product and/or an extension or amplification product of the probe or probe set.

In some embodiments, the decoy oligonucleotide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides in length. In some embodiments, the decoy oligonucleotide is no more than about 10, no more than about 15, no more than about 20, no more than about 25, no more than about 30, no more than about 35, no more than about 40, no more than about 45, no more than about 50, no more than about 60, no more than about 70, no more than about 80, no more than about 90, or no more than about 100 nucleotides in length. In some embodiments, the decoy oligonucleotide is shorter than the hybridization region of the probe or probe set and/or the target region in the target nucleic acid. In some embodiments, the decoy oligonucleotide is at least 1, 2, 3, 4, 5, 6, 7, 8, 9, 10, 11, 12, 13, 14, 15, 16, 17, 18, 19, 20, or more nucleotides shorter than the hybridization region of the probe or probe set and/or the target region in the target nucleic acid. In some embodiments, the decoy oligonucleotide is between 2 and 5 nucleotides shorter than the hybridization region and/or the target region. In some embodiments, the decoy oligonucleotide is between 5 and 10 nucleotides shorter than the hybridization region and/or the target region. In some embodiments, the decoy oligonucleotide is between 10 and 15 nucleotides shorter than the hybridization region and/or the target region.

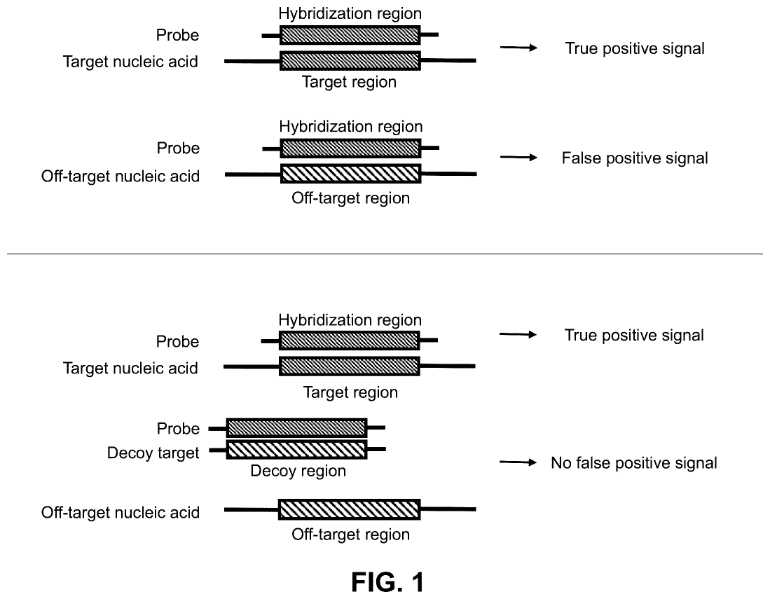

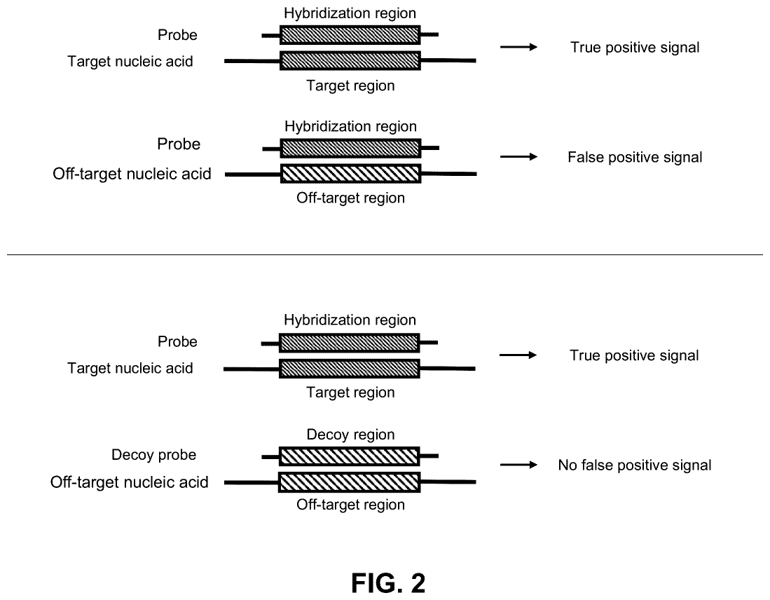

In some embodiments, the decoy oligonucleotide comprises a decoy region capable of hybridizing to a sequence of the hybridization region in the probe or probe set, for example, as shown in FIG. 1. In this case, the decoy oligonucleotide can be considered a decoy target, described in more detail in Section II.A.(i) below. In some embodiments, the decoy oligonucleotide comprises a decoy region capable of hybridizing to an off-target region and/or the target region in the biological sample, for example, as shown in FIG. 2. In this case, the decoy oligonucleotide can be considered a decoy probe, described in more detail in Section II.A.(ii) below.

### (i). Decoy Targets

In some aspects, disclosed herein is a method for analyzing a biological sample, comprising contacting the biological sample with a probe or probe set and a decoy oligonucleotide (e.g., a decoy target) in any suitable order, wherein the biological sample comprises a target nucleic acid comprising a target region, the probe or probe set comprises a hybridization region, and the decoy oligonucleotide comprises a decoy region capable of hybridizing to the hybridization region of the probe or probe set, and allowing the probe or probe set and the target nucleic acid to hybridize at one or more locations in the biological sample, wherein the decoy oligonucleotide reduces hybridization between the hybridization region and an off-target region in the biological sample. In some embodiments, the decoy oligonucleotide is a decoy target that hybridizes to the probe or probe set and competes with the real template (the target region) and/or with an off-target template (an off-target region) for hybridization to the probe or probe. In some embodiments, the decoy target is designed to avoid generating a product from the probe or probe set hybridized to the decoy target (e.g., to avoid generating a ligated probe and/or an amplification product using the decoy target as a ligation template and/or primer for amplification).

In some embodiments as shown in FIG. 1, top panel, in the absence of decoy target, the probe or probe set comprising a hybridization region capable of hybridizing to a target region may hybridize to an off-target region. In some embodiments, the hybridization region is 100% complementary to the target region, and between 80% and 99% complementary to the off-target region. As shown in the top panel of FIG. 1, hybridization to the off-target region can result in a false positive signal when the probe or probe set or a product thereof is detected (e.g., background or off-target signal (e.g., fluorescence) in a method comprising imaging the sample to detect an optical signal associated with the probe or probe set or a product thereof). As shown in the bottom panel of FIG. 1, in some embodiments the sample is contacted with a decoy target comprising a decoy region that hybridizes to the hybridization region of the probe or probe set. In some embodiments, the decoy region of the decoy target comprises or consists of the same sequence as an off-target region in the biological sample. In some embodiments, the decoy region of the decoy target has a different sequence than the off-target region. In some embodiments, the decoy region of the decoy target comprises one or more differences in sequence compared to the target region. In some instances, complementarity between the decoy region of the decoy target and the hybridization region of the probe or probe set is higher than complementarity between the hybridization region of the probe or probe set and the off-target region. In some instances, complementarity between the decoy region of the decoy target and the hybridization region of the probe or probe set is lower than complementarity between the hybridization region of the probe or probe set and the target region. As shown in FIG. 1, the decoy target can prevent or reduce hybridization of the probe or probe set to the off-target region, thus preventing or reducing the false positive signal. For example, the decoy target is designed to out-compete the off-target region for hybridization to the probe or probe set. In some embodiments, the off-target region is a region in an off-target nucleic acid (e.g., a different mRNA transcript from a target mRNA transcript). In some embodiments, in the presence of a decoy target comprising a decoy region that hybridizes to the hybridization region in the probe, no false positive signal or reduced false positive signal associated with the off-target region is produced (bottom panel of FIG. 1).

In some embodiments, the hybridization region has a higher sequence complementarity to the decoy region than to the off-target region. In some embodiments, the hybridization region has between 90% and 95%, 90% and 98%, 90% and 99%, 95% and 98%, and 95% and 99% sequence complementarity to the decoy region. In some embodiments, the hybridization region has less than any one of 99%, 98%, 97%, 96%, 95%, 90%, and 85% sequence complementarity to the off-target region. In some embodiments, the hybridization region has at least 95% sequence complementarity to the decoy region and less than 95% sequence complementarity to the off-target region. In some embodiments, the hybridization region has at least 99% sequence complementarity to the decoy region and between about 80% and about 95% sequence complementarity to the off-target region. In some embodiments, the hybridization region comprises at least 1, 2, 3, 4, 5, 6, 7, 8, 9, or 10 mismatches with the decoy region. In some embodiments, the decoy region comprises a sequence of the off-target region.

In some embodiments, the decoy region is shorter than the hybridization region. In some embodiments, the decoy region is at least 1, 2, 3, 4, 5, 6, 7, 8, 9, 10, or more than 10 nucleotides shorter than the hybridization region. In some embodiments, the decoy region is at least 10, at least 15, or It least 20 nucleotides shorter than the hybridization region. In some embodiments, the hybridization region is at least about 10, at least about 15, at least about 20, at least about 25, at least about 30, at least about 35, at least about 40, at least about 45, at least about 50, at least about 70, at least about 80, at least about 90, or at least about 100 nucleotides in length. In some embodiments, the hybridization region is between or between about any one of 5 and 200, 10 and 200, 15 and 200, 20 and 200, 5 and 100, 10 and 100, 15 and 100, 20 and 100, 5 and 50, 10 and 50, 15 and 50, 20 and 50, 5 and 20, or 10 and 40 nucleotides in length. In some embodiments, the decoy oligonucleotide is no more than about 10, no more than about 15, no more than about 20, no more than about 25, no more than about 30, no more than about 35, no more than about 40, no more than about 45, no more than about 50, no more than about 60, no more than about 70, no more than about 80, no more than about 90, or no more than about 100 nucleotides in length. In some embodiments, the decoy region between 5 and 100, between 5 and 50, between 5 and 20, between 10 and 100, between 10 and 50, between 10 and 20, or between 15 and 50 nucleotides in length.

In some embodiments, the decoy target is detectably labeled or not detectably labeled. In some embodiments, the decoy target is not detectably labeled. In some embodiments, the decoy target is detectably labeled. In some embodiments, the detectable label of the decoy target is used to remove the decoy target or a complex comprising the decoy target from the sample (e.g., by contacting the detectable label with a binding moiety that binds to the detectable label, optionally wherein the binding moiety is then removed from the sample). In some embodiments, the decoy target is directly labeled or indirectly labeled (e.g., by hybridization of a probe to an overhang region of the decoy target that does not hybridize to the hybridization region). In some embodiments, the probe hybridized to the decoy target is a bait probe that is detectably labelled with a moiety (e.g., a biotin moiety) for removing the decoy target from the sample. In other embodiments, upon hybridization the hybridization region of the probe or probe set, the decoy target does not comprise a region capable of directly or indirectly binding to a detectably labeled probe. In some embodiments, the decoy target does not comprise any overhang regions upon hybridization to the probe or probe set.

In some embodiments, upon hybridization to the hybridization region of the probe or probe set, the decoy target is not ligatable with itself, within the probe set, or with another oligonucleotide. For example, the decoy target may lack a 5′ phosphate group, may comprise a 5′-OMe-dT group, or may comprise a 3′ dideoxynucleotide, or any other moiety or modification that blocks ligation. In some embodiments, upon hybridization to the hybridization region of the probe or probe set, the decoy target is not detectable by detectable probes configured to detect the probe or probe set or product thereof. In some embodiments, upon hybridization to the hybridization region of the probe or probe set, the decoy target is not capable of generating a product that is detectable by detectable probes configured to detect the probe or probe set or product thereof. In some embodiments, the decoy target is not capable of being used as a primer for extension of the decoy target (e.g., using the probe or probe set as a template). In some instances, the decoy target comprises a 3′ chain terminating nucleotide or modification such as a 3′ dideoxynucleotide, a 2′,3′-dideoxynucleoside or a 3′-deoxynucleoside.

In some embodiments, the decoy target comprises one or more modifications that facilitate removal of a complex comprising the decoy target from a biological sample. In some embodiments, the modification is a biotin modification, a dual-biotin modification, a triple-biotin modification, a desthiobiotin modification, or an iminobiotin modification. In some embodiments the modification facilitates removal of the complex comprising the decoy target from a biological sample using streptavidin (e.g., streptavidin-conjugated beads). Other decoy target modifications comprising binding moieties can be used in combination with a suitable binding partner for selective removal decoy targets and complexes comprising a decoy target hybridized to a probe or probe set. Exemplary modifications include a FITC modification (recognized by anti-FITC for removal). In some embodiments wherein the probe or probe set is ligated using the target nucleic acid as a template, a complex comprising the decoy target hybridized to the probe or probe set is removed from the sample prior to ligation. In other embodiments, the complex comprising the decoy target hybridized to the probe or probe set is removed from the sample after ligation. In some instances, the complex comprising the decoy target hybridized to the probe or probe set is removed from the sample prior to detecting a signal associated with the probe or probe set or a product thereof at one or more locations in the sample (e.g., prior to imaging the sample). In some embodiments, the complex comprising the decoy target hybridized to the probe or probe set is removed from the sample between the ligating of the probe or probe set and performing rolling circle amplification of the circularized probe or probe set. In some embodiments, the probe or probe set hybridization and ligation are carried out simultaneously and/or under the same reaction condition. In some embodiments, the hybridization and ligation are not separated by a wash step. In these cases, the complex comprising the decoy target hybridized to the probe or probe set can be removed from the sample after ligation and prior to detecting the ligated probe or probe set.

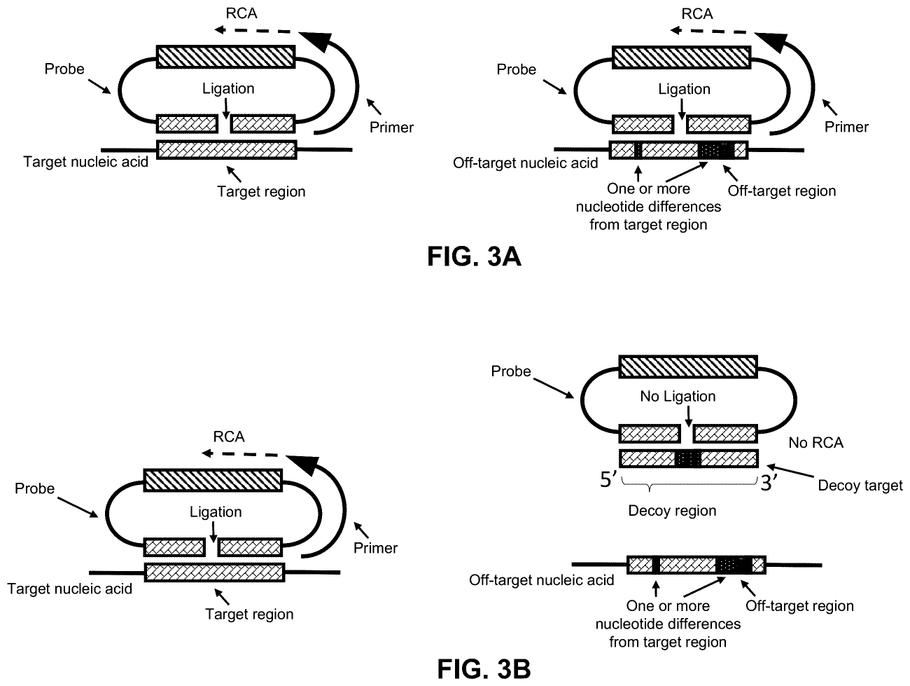

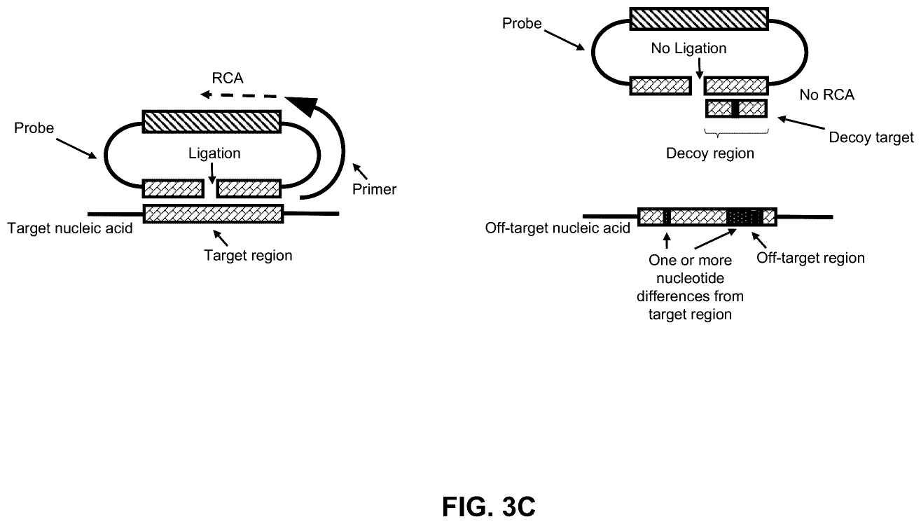

In some embodiments, the probe or probe set is ligatable using the target region as template. The ligated product may be a linear ligated probe formed from a first probe and a second probe. In some embodiments, the ligated product is a circular probe, as shown in FIG. 3A. The circularized probe can be amplified by rolling circle amplification (RCA), and the RCA product can be detected in the sample. However, hybridization of the probe or probe set to an off-target region (e.g., a region comprising one or more nucleotide differences from the target region) may result in ligation of the probe or probe set, production of an RCA product, and detection of a false positive signal from the RCA product (FIG. 3A, right panel). In some embodiments, a decoy target is used to block hybridization of the probe or probe set to the off-target region, while avoiding ligation of the probe or probe set using the decoy target as a template.

In some embodiments, the probe or probe set is ligatable using the target region as a template, but the decoy target serves as a poor template for ligation. In some embodiments, the decoy target comprises one or more mismatches with the probe or probe set at or near a ligation point of the probe or probe set (for example, as shown in FIG. 3B, right panel), thus preventing or reducing the likelihood that the probe or probe set will be ligated using the decoy target as a template. In some embodiments, the decoy region of the decoy target hybridizes only to one portion of a split hybridization region (e.g., does not bridge the split hybridization region) and cannot serve as a template for ligation, as shown in FIG. 3C (right panel). In some embodiments, the decoy region of the decoy probe can be a split region that does not bridge the split hybridization region of a probe or probe set. For example, the decoy probe can be provided in two or more parts that separately hybridize to a first and second end of a circularizable probe, or that separately hybridize to a first probe and second probe of a ligatable probe set. Thus, the split decoy region may not be able to serve as a template for ligation of the ends of the probe or probe set. As shown in FIG. 3C, the decoy target can block hybridization of at least one portion of a split hybridization region to an off-target region. The decoy region can be designed to hybridize more stably to the portion of the split hybridization region than the off-target region, but less stably than the target region. Thus, in some embodiments the portion of the split hybridization bound by the decoy target will be able to bind to the target region but binds less efficiently or does not bind to the off-target region. For example, the decoy target may hybridize to only one end of a circularizable probe, or a decoy target may hybridize to only one of a first and second probe. As shown in the left panels of FIG. 3B and FIG. 3C, the target region can outcompete the decoy target and/or displace the decoy target from the probe or probe set, allowing ligation of the probe or probe set templated by the true target region.

In some embodiments, the decoy target is not extendable by a polymerase. Thus, in some embodiments, the decoy target cannot function as a primer for amplification (e.g., rolling circle amplification) of the probe or probe set. In the case of a circularizable probe or probe set, preventing the decoy target from functioning as a primer can reduce the likelihood that a probe or probe set hybridized to a decoy target rather than the target region will generate a rolling circle amplification product. In some embodiments, the decoy target comprises one or more mismatches with the probe or probe set at or near a ligation point of the probe or probe set, and further comprises a modification or blocking moiety that prevents extension of the decoy target by a polymerase. In some embodiments, the decoy target lacks a 3′ hydroxyl group. In some embodiments, the decoy target comprises a 3′ dideoxynucleotide. In some embodiments, the decoy target comprises a 3′ stem-loop structure that prevents the 3′ end from being used as a primer. In some embodiments, the decoy target can also comprise one or more modifications that facilitate removal of a complex comprising the decoy target from a biological sample as described above. Thus, even if the probe or probe set is ligated using the decoy target as a template, it may be removed from the sample to prevent detecting the ligated probe from the decoy target or a product thereof.

In some embodiments, the decoy target and the probe or probe set are mixed prior to contacting the biological sample. In some embodiments, the decoy target and the probe or probe set are contacted with the biological sample in the same solution. In some embodiments, the decoy target is provided to a biological sample in a hybridization complex with the probe or probe set. In some embodiments, the method comprises allowing the decoy target and the probe or probe set to hybridize together prior to contacting the sample. In other embodiments, the decoy target is provided to a biological sample separately from a probe or probe set. In some embodiments, the decoy target is contacted with the biological sample prior to contacting the sample with the probe or probe set. In some embodiments, the decoy target is contacted with the sample after contacting the sample with the probe or probe set. In some embodiments, the probe or probe set is a ligatable probe or probe set and the decoy target is contacted with the biological sample prior to contacting the sample with a ligase. In some embodiments, the probe or probe set is a ligatable probe or probe set and the decoy target and probe or probe set are contacted with the sample together with the ligase, optionally wherein the decoy probe and probe or probe set are pre-hybridized. In some embodiments, the ligatable probe or probe set can be a circularizable probe or probe set, or a probe set comprising a first and second probe that can be ligated using the target nucleic acid as a template to generate a ligated first-second probe.

In some embodiments, the decoy target and the probe or probe set are provided to the biological sample at a 1:1 ratio (e.g., a 1:1 molecular ratio). In some embodiments, the decoy target and the probe or probe set are provided to the biological sample at a molecular ratio of at least 1.1:1, 1.2:1, 1.3:1, 1.5:1, 2:1, 2.5:1, 3:1, 5:1, 10:1, 20:1, 50:1, or 100:1. In some embodiments, the decoy target and the probe or probe set are provided to the biological sample at a ratio of no more than 1.3:1, 1.5:1, 2:1, 2.5:1, 3:1, 5:1, 10:1, 20:1, 50:1, 100:1, or 200:1.

### (ii). Decoy Probes

In some aspects, disclosed herein is a method for analyzing a biological sample, comprising contacting the biological sample with a probe or probe set and a decoy oligonucleotide (e.g., a decoy probe) in any suitable order, wherein the biological sample comprises a target nucleic acid comprising a target region, the probe or probe set comprises a hybridization region, and the decoy oligonucleotide comprises a decoy region capable of hybridizing to an off-target region of a nucleic acid molecule in the biological sample (e.g., an off-target nucleic acid molecule), and allowing the probe or probe set and the target nucleic acid to hybridize at one or more locations in the biological sample, wherein the decoy oligonucleotide reduces hybridization between the hybridization region and the off-target region in the biological sample. In some embodiments, the decoy oligonucleotide is a decoy probe that hybridizes to an off-target region and/or to the target region. In some embodiments, the decoy oligonucleotide hybridizes to an off-target region with more stability than to the target region. In some embodiments, the probe or probe set displaces the decoy oligonucleotide from the target region but not from an off-target region. In some embodiments, the probe or probe set out-competes the decoy oligonucleotide for hybridization to the target region, but does not out-compete the decoy oligonucleotide for hybridization to an off-target region. For example, the decoy region has a lower sequence complementarity to the target region compared to the sequence complementarity of the hybridization region of the probe or probe set to the target region. In some embodiments, the decoy oligonucleotide is a decoy probe that hybridizes to an off-target region or to the target region but is not detectable or does not generate a signal in downstream steps of the assay.

In some embodiments as shown in FIG. 2, top panel, in the absence of a decoy probe, the probe or probe set comprising a hybridization region capable of hybridizing to a target region may hybridize to an off-target region. In some embodiments, the hybridization region is 100% complementary to the target region, and between 80% and 99% complementary to the off-target region. As shown in the top panel of FIG. 2, hybridization to the off-target region can result in a false positive signal when the probe or probe set or a product thereof is detected (e.g., background or off-target fluorescence in a method comprising imaging the sample to detect an optical signal associated with the probe or probe set or a product thereof). As shown in the bottom panel of FIG. 2, in some embodiments the sample is contacted with a decoy probe comprising a decoy region that hybridizes to the off-target region. Although not shown in the figure, in some embodiments the decoy probe can also hybridize to the target region. In some embodiments, the decoy region of the decoy probe has higher complementarity to the off-target region than to the target region. In some embodiments, the complementarity between the hybridization region of the probe or probe set is higher than the complementarity between the decoy probe and the target region. As shown in the bottom panel of FIG. 2, the decoy probe can prevent or reduce hybridization of the probe or probe set to the off-target region, thus preventing or reducing the false positive signal. In some embodiments, the off-target region is a region in an off-target nucleic acid (e.g., a different mRNA transcript from a target mRNA transcript). In some embodiments, in the presence of a decoy probe comprising a decoy region that hybridizes to the off-target region, no false positive signal is produced (bottom panel of FIG. 2).

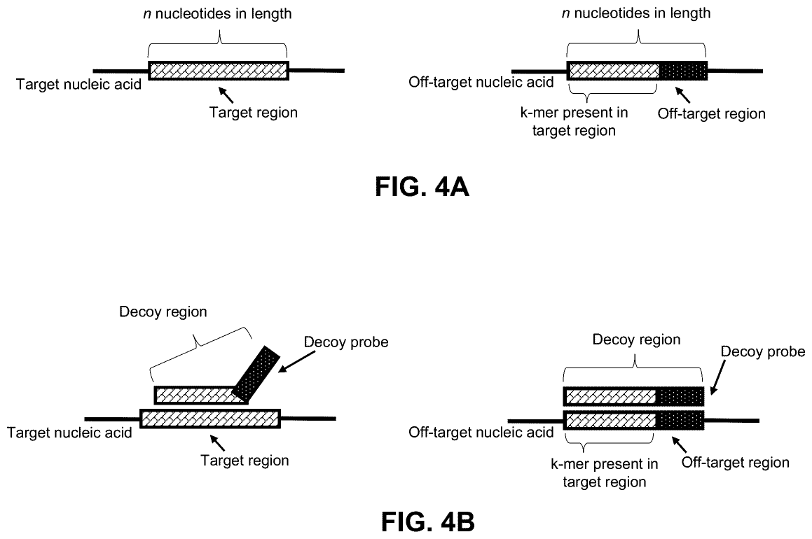

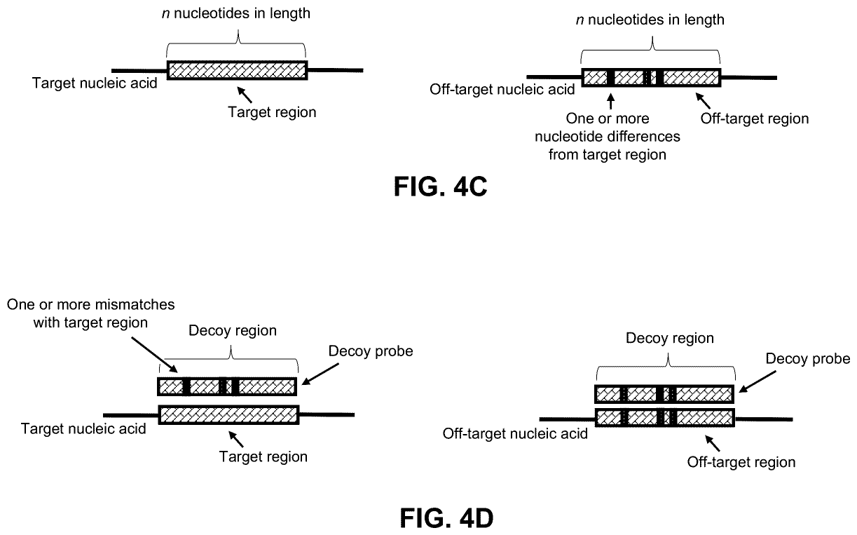

In some embodiments, the decoy probe does not comprise any overhang regions when hybridized to the off-target region (e.g., the decoy probe does not comprise a 5′ or 3′ region that does not hybridize to the nucleic acid comprising the off-target region). In some embodiments, the decoy probe comprises an overhang region when hybridized to the target region, but not when the hybridized to the off-target region. For example, a portion of the decoy region may hybridize to the off-target region, but not to the target region. Thus, in some embodiments the probe or probe set can displace the decoy probe from the target region but not from the off-target region. As shown in FIG. 4A, in some cases a given target region may comprise one or more k-mers that are present in off-target nucleic acids. For example, a target region of n nucleotides in length may be specific (e.g., unique) to a target nucleic acid among the nucleic acids present in a biological sample, but may comprise a k-mer that is repeated in one or more other nucleic acid molecules. If the k-mer is of sufficient length, it may result in off-target hybridization of a probe or probe set. In some embodiments, an off-target region is identified as comprising a k-mer that is present in the target region, as shown in FIG. 4A. A decoy probe can be designed with a decoy region complementary to the repeated k-mer and to an additional sequence adjacent to the k-mer (dark region in FIG. 4A), which together can form the off-target region. Because the decoy region hybridizes to the full off-target region (including the additional sequence) but only to the k-mer present in the target region, it hybridizes to the off-target region more stably than to the target region (FIG. 4B). In some embodiments, the probe or probe set hybridizes to the target region in the presence of the decoy probe but not to the off-target region. In some embodiments, the overhang region present when the decoy probe is hybridized to the target region does not hybridize to any detectable probes that are contacted with the sample.

As shown in FIG. 4C, in some embodiments an off-target region is a region of the same length as the target region but comprising one or more nucleotide differences from the target region. In some embodiments, the off-target region differs from the target region by a single nucleotide. In some embodiments, the off-target region differs from the target region by at least any one of 2, 3, 4, 5, 6, 7, 8, 9, 10, 15, 20, 25, or 30 nucleotides. In some embodiments, the target region differs from the target region by no more than 20, 15, 10, 9, 8, 7, 6, 5, 4, 3, or 2 nucleotides. In some embodiments, as shown in FIG. 4D, the decoy probe comprises a decoy region that is complementary to the off-target region. Thus, in some embodiments, the decoy region comprises one or more mismatches with the target region, wherein the number of mismatches with the target region corresponds to the number of differences between the off-target region and the target region. In some embodiments, the decoy probe is not 100% complementary to the off-target region.

In some embodiments, one or more off-target regions are known or are identified as being present in the biological sample (for example, using sequence alignment tools to identify off-target regions with sequence homology to a target region, as described above). In some embodiments, a decoy probe is provided for a known or predicted off-target region for a given probe or probe set. In some embodiments the decoy probe comprises a decoy region designed to hybridize to the known off-target region. In some embodiments, the method comprises contacting the sample with a plurality of decoy probes designed to hybridize to a plurality of off-target regions.

In some embodiments, the off-target region may not be known or identified a priori. In some aspects, provided herein is a method for analyzing a biological sample comprising contacting the biological sample with a probe or probe set and a decoy probe in any suitable order, wherein the biological sample comprises a target nucleic acid comprising a target region, the probe or probe set comprises a hybridization region and the decoy probe comprises a decoy region capable of hybridizing to the target region, wherein the decoy region hybridizes to the target region less stably than the probe or probe set. In some embodiments, the decoy probe competes with the probe or probe set for hybridization to the target region. In some embodiments, the decoy region of the decoy probe has less than 99%, less than 98%, less than 97%, less than 96%, less than 95%, less than 90%, less than 85%, or less than 80% sequence identity with the probe or probe set. In some embodiments, the decoy region of the decoy probe has less than 99%, less than 98%, less than 97%, less than 96%, less than 95%, less than 90%, less than 85%, or less than 80% sequence identity with the hybridization region of the probe or probe set. In some embodiments, the decoy region of the decoy probe has at least 97%, at least 96%, at least 95%, at least 90%, at least 85%, at least 80%, or at least 70% sequence identity with the probe or probe set. In some embodiments, the decoy region of the decoy probe has at least 97%, at least 96%, at least 95%, at least 90%, at least 85%, at least 80%, or at least 70% sequence identity with the hybridization region of the probe or probe set. In some embodiments, the sequence differences between the decoy probe and the probe or probe set are randomly distributed in the decoy probe. In some embodiments, one or more of the sequence differences between the decoy probe and the probe or probe set are at the 5′ end of the decoy probe or the 3′ end of the decoy probe. In some embodiments, the probe or probe set is a ligatable probe or probe set. In some embodiments, provided herein is a method for analyzing a biological sample comprising contacting the biological sample with a probe or probe set and a plurality of decoy probes in any suitable order, wherein the biological sample comprises a target nucleic acid comprising a target region, the probe or probe set comprises a hybridization region and each decoy probe of the plurality comprises a decoy region capable of hybridizing to the target region, wherein the decoy region hybridizes to the target region less stably than the probe or probe set, and wherein each decoy probe of the plurality comprises a different mismatch or combination of mismatches with the target sequence. In some embodiments, the decoy region of each decoy probe of the plurality of decoy probes has less than 99%, less than 98%, less than 97%, less than 96%, less than 95%, less than 90%, less than 85%, or less than 80% sequence identity with the probe or probe set.

In some embodiments, a plurality of decoy probes can be contacted with the biological sample, wherein the plurality of decoy probes comprises different decoy probes with different levels of sequence identity to the probe or probe set. In some embodiments, the different decoy probes are contacted with the biological sample at different concentrations. In some embodiments, the method comprises contacting the sample with a first decoy probe with a decoy region having a first percent sequence identity to the hybridization region of the probe or probe set at a first concentration and a second decoy probe having a decoy region with a second percent sequence identity to the hybridization region of the probe or probe set at a second concentration. In some embodiments, the first decoy probe comprises a decoy region with a higher sequence identity to the hybridization region of the probe or probe set than that of the second decoy probe. In some embodiments, the first decoy probe is contacted with the sample and a lower concentration than the second decoy probe. In some embodiments, the method comprises contacting the sample with a first decoy probe at a first concentration, a second decoy probe at a second concentration, and a third decoy probe at a third concentration. In some instances, the first decoy probe, second decoy probe, and third decoy probe are for hybridizing to the same off-target region or at least a portion thereof. In some embodiments, up to 4, 5, 6, 7, 8, 9, 10, or more different decoy probes are contacted with the sample, wherein each of the different decoy probes comprises a decoy region having a different percent sequence identity to the hybridization region of the probe or probe set. In some embodiments, each of the different decoy probes is contacted with the sample at a different concentration. In some embodiments, decoy probes comprising decoy regions having more mismatches with the target region are contacted with the sample at higher concentrations, and decoy probes having decoy regions having fewer mismatches with the target region are contacted with the sample at lower concentrations.

In some embodiments, the decoy region of decoy probe herein does not comprise mismatches with the target region. In some embodiments, the decoy probe comprises a decoy region having 100% complementarity to the target region. In some embodiments, the probe or probe set is contacted with the sample at a higher concentration than the decoy probe. In some embodiments, the probe or probe set and the decoy probe are contacted with the sample at a ratio of at least about 100:1, 50:1, 40:1, 30:1, 20:1, 15:1, 10:1, 5:1, or 2:1 probe or probe set to decoy probe. In some embodiments, the probe or probe set and the decoy probe are contacted with a sample at a ratio between about 2:1 and about 5:1, about 2:1 and about 10:1, about 5:1 and about 20:1, and about 10:1 and about 100:1 probe or probe set to decoy probe.

In some embodiments, the probe or probe set is a ligatable probe comprising a split hybridization region, wherein the split hybridization region can be ligated using the target region as a template to produce a ligated probe. The ligated probe can be a circularized probe (e.g., wherein the probe or probe set is a circularizable probe or probe set), or the ligated probe can be a linear probe (e.g., a ligated first-second probe generated from a first probe and a second probe). In some embodiments, the probe or probe set is a ligatable probe set and the decoy probe is not a ligatable probe. In some embodiments, the hybridization region of the probe or the probe set is a split hybridization region for templated ligation, but the decoy region of the decoy probe is not a split hybridization region. In other embodiments, the decoy probe can comprise a split hybridization region. In some embodiments, the decoy probe comprises a split hybridization region, wherein the decoy probe comprises one or more modifications that block ligation. In some embodiments, upon hybridization to a target region or an off-target region, the decoy probe is not ligatable with itself, within the probe set, or with another oligonucleotide. In some embodiments, the decoy probe lacks a phosphate group at the 5′ end. In some embodiments, the decoy region of the decoy probe is a split hybridization region comprising a gap between a first probe and a second probe, wherein the 3′ end of the first probe comprises is not extendable by a polymerase to fill the gap for ligation of the decoy region. In some embodiments, the decoy probe lacks a 3′ hydroxyl group. In some embodiments, the decoy probe is not extendable by a polymerase. In some embodiments, the decoy probe comprises an irreversible terminating group, such as a 3′ dideoxynucleotide or any other chain terminator.

In some embodiments, upon hybridization to an off-target region or a target region, the decoy probe does not comprise a region capable of directly or indirectly binding to a detectably labeled probe. In some embodiments, upon hybridization to an off-target region or a target region, the decoy probe is not detectable by detectable probes configured to detect the probe or probe set or product thereof. In some embodiments, upon hybridization to an off-target region or a target region, the decoy probe is not capable of generating a product that is detectable by detectable probes configured to detect the probe or probe set or product thereof. In some embodiments, the decoy probe comprises one or more modifications (e.g., modified nucleotides comprising bulky sugar, backbone, or residue modifications) that reduce its ability to be used as a template for amplification.

In some embodiments, the decoy probe is a modified version of the probe or probe set that is not capable of or ligating within itself or to another probe, but is otherwise identical to the probe or probe set. In some embodiments, the decoy probe is a modified version of the probe or probe set that is not capable of ligating within itself or to another probe and/or is not capable of serving as a template for amplification, but is otherwise identical to the probe or probe set. In some embodiments, the decoy probe is a modified version of the probe or probe set comprising any one of the following modifications relative to the probe or probe set: (i) is not capable of ligating within itself or to another probe, (ii) is not capable of serving as a template for amplification, (iii) does not comprise a region capable of directly or indirectly binding to a detectably labeled probe, (iv) is not detectable by detectable probes configured to detect the probe or probe set or product thereof (e.g., does not comprise a barcode sequence or complement thereof wherein the method comprises contacting the sample with detectable probes to detect the barcode sequence or a complement thereof in the probe or probe set or a product thereof). In some embodiments, the decoy probe is a circularizable probe or probe set. In some embodiments, the decoy probe comprises a first probe and a second probe that together comprise a split hybridization region.

In some aspects, provided herein is a method for analyzing a biological sample, comprising: contacting the biological sample with a circularizable probe or probe set, and a decoy probe in any suitable order, wherein: the biological sample comprises a target nucleic acid comprising a target region, the circularizable probe or probe set comprises a first hybridization region and a second hybridization region which, upon hybridization to the target region, are ligatable, and the decoy probe comprises a decoy region capable of hybridizing to the target region. In some embodiments, the decoy region is a split hybridization region configured to hybridize such that the ends of the decoy probe are juxtaposed (e.g., in a similar configuration to the circularizable probe or probe set), but the decoy probe lacks a 5′ phosphate. In some embodiments, the decoy probe is not ligatable. In some embodiments, the method comprises allowing the circularizable probe or probe set and the target nucleic acid to hybridize at one or more locations in the biological sample. In some embodiments, the decoy probe reduces hybridization between the first and/or second hybridization regions and an off-target region in the biological sample. In some embodiments, the method comprises circularizing the circularizable probe or probe set to generate a circular probe by ligating the first and second hybridization regions using the target region as template, with or without flap cleavage and with or without gap filling prior to ligation. In some embodiments, the decoy probe is not circularized. In some embodiments, the method comprises generating a rolling circle amplification (RCA) product of the circular probe; and detecting a signal associated with the RCA product at the one or more locations, thereby detecting the target nucleic acid in the biological sample.

In some embodiments, the target region and/or the off-target region is pre-hybridized to the decoy probe. In some embodiments, the off-target region is pre-hybridized to the decoy probe. In some embodiments, the target region and the off-target region are pre-hybridized to the decoy probe. In some embodiments, the method comprises contacting the sample with the decoy probe prior to contacting the sample with the probe or probe set. In some embodiments, the method comprises contacting the sample with the decoy probe at least 5, 10, 15, 20, 30, 60, 80, 100, or 120 minutes prior to contacting the sample with the probe or probe set. In some embodiments, the method comprises incubating the sample with the decoy probe prior to contacting the sample with the probe or probe set.

### B. Probes or Probe Sets

Any suitable probe design can be combined with a decoy oligonucleotide in accordance with the methods described herein. The decoy oligonucleotide can be a decoy target or a decoy probe. Exemplary barcoded probes or probe sets may comprise a circularizable probe or probe set (e.g., based on a padlock probe, a gapped padlock probe, a SNAIL (Splint Nucleotide Assisted Intramolecular Ligation) probe set), a PLISH (Proximity Ligation in situ Hybridization) probe set, a RollFISH probe set, or a PLAYR (Proximity Ligation Assay for RNA) probe set). In some embodiments, an exemplary barcoded probe or probe set is not circular or circularizable. Examples of barcoded probes or probe sets include, but are not limited to, L-shaped probes (e.g., a probe comprising a target-hybridizing sequence and a 5′ or 3′ overhang upon hybridization to its target sequence), or U-shaped probes (e.g., a probe comprising a target-hybridizing sequence and a 5′ overhang and a 3′ overhang upon hybridization to its target sequence). The specific probe or probe set design can vary.

In some embodiments, the probe or probe set is a probe comprising a 3′ or 5′ overhang upon hybridization to the target nucleic acid (e.g., an L-shaped probe). In some embodiments, the 3′ or 5′ overhang comprises one or more detectable labels and/or barcode sequences. In some embodiments, multiple L-shaped probes are hybridized to a plurality of target regions within a particular target nucleic acid molecule (e.g., tiling across multiple regions in the target nucleic acid molecule). Such tiling of probes can provide signal amplification by increasing the number of detectable labels and/or barcode sequences per target nucleic acid. For example, between 10 and 20, between 10 and 30, or between 20 and 40 probes can be hybridized per target nucleic acid molecule. In some embodiments according to the methods described herein, each target nucleic acid molecule can be hybridized by no more than 1, 2, 3, 4, 5, 6, 7, 8, 9, or 10 probes such as L-shaped probes. In some embodiments, a single probe is hybridized per target nucleic acid molecule (e.g., per target RNA). In some embodiments, the decoy oligonucleotide (e.g., a decoy target or a decoy probe) reduces off-target hybridization of the probe, thus allowing detection of a single short target region (e.g., between 10 and 100 nucleotides in length, between 10 and 50 nucleotides in length, or between 15 and 30 nucleotides in length) by a single L-shaped probe without substantial loss of specificity. Any suitable method of signal amplification can be used to detect a barcode sequence in the overhang region of the L-probe.

In some embodiments, the probe or probe set is a probe comprising a 3′ overhang and a 5′ overhang upon hybridization to the target nucleic acid. In some embodiments, the 3′ overhang and the 5′ overhang each independently comprises one or more detectable labels and/or barcode sequences. In some embodiments, multiple U-shaped probes are hybridized to a plurality of target regions within a particular target nucleic acid molecule (e.g., tiling across multiple regions in the target nucleic acid molecule). Such tiling of probes can provide signal amplification by increasing the number of detectable labels and/or barcode sequences per target nucleic acid. For example, between 10 and 20, between 10 and 30, or between 20 and 40 probes can be hybridized per target nucleic acid molecule. In some embodiments according to the methods described herein, each target nucleic acid molecule can be hybridized by no more than 1, 2, 3, 4, 5, 6, 7, 8, 9, or 10 probes such as U-shaped probes. In some embodiments, a single probe is hybridized per target nucleic acid molecule (e.g., per target RNA). In some embodiments, the decoy oligonucleotide (e.g., a decoy target or a decoy probe) reduces off-target hybridization of the probe, thus allowing detection of a single short target region (e.g., between 10 and 100 nucleotides in length, between 10 and 50 nucleotides in length, or between 15 and 30 nucleotides in length) by a single U-shaped probe without substantial loss of specificity. Any suitable method of signal amplification can be used to detect a barcode sequence in the first and/or second overhang region of the U-probe.

In some embodiments, the probe or probe set is a circular probe. In some embodiments, the probe or probe set is a circularizable probe or probe set. In some embodiments, the probe or probe set is designed for RNA-templated ligation. See, e.g., U.S. Pat. Pub. 2020/0224244 which is hereby incorporated by reference in its entirety. In any of the embodiments herein, the circularizable probe or probe set can comprise one, two, three, four, or more ribonucleotides. In some embodiments, the circularizable probe or probe set is designed to be circularized using the target nucleic acid (e.g., a DNA or RNA target nucleic acid) as a template. In some embodiments, the circularizable probe or probe set is designed to be circularized using another probe as a template (e.g., as in the case of SNAIL or RollFISH probes). In some embodiments, the probe used as a template for circularization is also used as a primer for amplification of the circularized probe or probe set. In some embodiments, a separate primer is provided for amplification of the circularized probe or probe set. In some embodiments, the decoy oligonucleotide (e.g., decoy target) is a poor template for ligation of the circularizable probe or probe set (e.g., when the decoy oligonucleotide is designed to hybridize to the probe or probe set, it may comprise one or more mismatches near the ligation point of the probe or probe set, as described in Section II.A above). In some embodiments, the decoy oligonucleotide (e.g., decoy target) is not capable of serving as a primer for amplification of a circular or circularizable probe (e.g., when the decoy oligonucleotide is designed to hybridize to the probe or probe set, it may comprise a 3′ end that cannot be extended by a polymerase, such as a 3′ dideoxynucleotide or any other chain terminator, as described in Section II.A above). Any other modifications or variations of circularizable probe or probe sets can be used.

In some embodiments, the probe or probe set comprises a primer binding site. In some embodiments, a primer is provided for hybridization to the primer binding site, wherein the primer can be extended to form an amplification product of the probe or probe set (e.g., a rolling circle amplification product of a circular or circularized probe). A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the probe or probe set comprises a first probe and a second probe that can be ligated to generate a ligated first-second probe (e.g., a linear ligated probe). In some embodiments, a linear ligated probe can be circularized using an additional bridge probe that is ligated to either end of the ligated linear probe (e.g., in a templated or non-templated ligation). In some embodiments, the first and/or second probe comprises an overhang region, which may optionally comprise one or more barcode sequences for detection of the first and/or second probe or the ligated first-second probe. In some embodiments, the probe or probe set is a circularizable probe or probe set (e.g., a padlock probe). In some embodiments, the circularizable probe or probe set comprises one or more barcode sequences for detection of circularizable probe or probe set, the circularized probe or probe set, or an amplification product thereof.

In some embodiments, a probe or probe set disclosed herein can comprise one, two, three, four, or more ribonucleotides in a DNA backbone. In any of the embodiments herein, the one or more ribonucleotides can be at and/or near a ligatable 3′ end of the circularizable probe or probe set. The probe or probe may comprise an optional 3′ RNA base. In some embodiments, a probe or probe set disclosed herein can comprise a 5′ flap which may be recognized by a structure-specific cleavage enzyme (e.g. an enzyme capable of recognizing the junction between single-stranded 5′ overhang and a DNA duplex and cleaving the single-stranded overhang). In some embodiments, the flap is positioned between a 3′ end and 5′ end of a split hybridization region upon hybridization of the probe to the target region, and cleavage of the flap allows ligation of the 3′ end to the 5′ end of the split hybridization region. Methods of ligating a first and second hybridization region with or without flap cleavage are described in U.S. Pat. Pub. 20200224244, the entire content of which is herein incorporated by reference. In some embodiments, the decoy oligonucleotide is a poor template for ligation of the first and second probe (e.g., when the decoy oligonucleotide is designed to hybridize to the probe or probe set, it may comprise one or more mismatches near the ligation point of the probe or probe set, as described in Section II.A above).

In some embodiments, the probe or probe set comprises a split hybridization region configured to hybridize to a splint. In some embodiments, the split hybridization region comprises one or more barcode sequences. For example, a probe set can comprise two probes that hybridize to adjacent portions of the target region, wherein each probe comprises an overhang region that does not hybridize to the target nucleic acid. The overhang regions can together form a split-hybridization region, either in a double “Z”-like configuration or a double “U”-like configuration. The split hybridization region can comprise one or more barcode sequences specific to the target region, so that the target region can be identified by hybridizing a detectable splint to the split hybridization region. The splint may be directly or indirectly labeled. In some embodiments, the splint is a bridge probe. In some embodiments, the splint is ligated to one or more other probes (e.g., to form a circularized probe), and optionally amplified by rolling circle amplification. In some embodiments, the splint comprises a barcode sequence (e.g., in an overhang region) that can be detected using any of the signal amplification and detection methods described herein, such as assembly of branched DNA structures, HCR, LO-HCR, RCA, PER, etc. Examples of probes or probe sets comprising split hybridization regions (e.g., Z-probes, proximity ligation in situ hybridization (PLISH) probes, or split-FISH probes) have been described, for example, in U.S. Pat. Pub. 20160115555, U.S. Pat. Pub. US20200224243, U.S. Pat. Pub. 20160108458, US20230083623, and WO2021/167526, the contents of each of which are herein incorporated by reference in their entireties.

In some embodiments described herein, the probe or probe set comprises a hybridization region (optionally a split hybridization region) capable of hybridizing to a target region in or associated with an analyte in a biological sample. In some embodiments, the hybridization region is complementary to the target region. In some embodiments, the hybridization region is at least about 10, at least about 15, at least about 20, at least about 25, at least about 30, at least about 35, at least about 40, at least about 45, at least about 50, at least about 70, at least about 80, at least about 90, or at least about 100 nucleotides in length. In some embodiments, the hybridization region is between or between about any one of 5 and 200, 10 and 200, 15 and 200, 20 and 200, 5 and 100, 10 and 100, 15 and 100, 20 and 100, 5 and 50, 10 and 50, 15 and 50, 20 and 50, 5 and 20, or 10 and 40 nucleotides in length. In some embodiments, the hybridization region is a split hybridization region.

In some embodiments, the split hybridization region comprises a first hybridization region and a second hybridization region. In some embodiments, the first hybridization region is at a first end of a probe or probe set and the second hybridization region is at a second end of a probe or probe set. In some embodiments, the first hybridization region is at a 3′ end of a probe or probe set and the second hybridization region is at a 5′ end of a probe or probe set, or vice versa. In some embodiments, the first and second hybridization regions are at a first and second end of a circularizable probe. In some embodiments, the first and second hybridization regions are in a first and second probe. In some embodiments, the 3′ or 5′ end of the probe or probe set comprises a flap (e.g., an overhang region that does not hybridize to the target nucleic acid) that is cleaved prior to ligation of the probe or probe set. In some embodiments, the first hybridization region and the second hybridization region are independently between or between about any one of 5 and 200, 10 and 200, 15 and 200, 20 and 200, 5 and 100, 10 and 100, 15 and 100, 20 and 100, 5 and 50, 10 and 50, 15 and 50, 20 and 50, 5 and 20, or 10 and 40 nucleotides in length.

In some embodiments, the probe or probe set comprises an anchor sequence, which can be a common sequence among a plurality of probes or probe sets for a plurality of target regions. In some embodiments, the method comprises contacting the sample with an anchor probe configured to hybridize to the anchor sequence or a complement thereof. In some embodiments, the anchor probe is complementary to the anchor sequence or complement thereof. In some embodiments, the anchor probe is a detectable probe. The anchor probe can be directly labeled or indirectly labeled (e.g., by direct or indirect hybridization of one or more detectably labeled probes to the anchor probe). In some embodiments, the method comprises imaging the sample to detect hybridization of the anchor probe, thereby detecting a plurality of analytes simultaneously.

In some embodiments, the target region is a marker sequence for a particular analyte, which identifies the particular analyte (e.g., alone or in combination with one or more other marker sequences). Thus, in some embodiments, a target region for a given target analyte is specific to that analyte, or unique, such that multiple target analytes can be distinguished from each other. In some embodiments, the analyte is an RNA molecule (e.g., an endogenous RNA molecule). Various analytes that may comprise target regions, and methods of associating target regions with different analytes, are described in Section VII.B below.

In some embodiments, the target region is present in a group of related molecules, e.g. isoforms or variants or mutants of an RNA transcript for a given gene. In some embodiments, the target region is specific to a particular subset of molecules (e.g., specific to a particular variant or mutant of an endogenous analyte such as an RNA molecule. For example, in some embodiments, the target region comprises a particular single nucleotide variant. In some embodiments, the target region may be unique or specific to the particular variant. In this way different variants, or isoforms, or mutants may be identified or distinguished from one another using the probes or probe sets and decoy oligonucleotides described herein.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte or labeling agent (e.g., any of products described in Section B (iii) above) as a tag or identifier (ID) sequence (e.g. a barcode) for the analyte or labeling agent. It may thus be a synthetic or artificial sequence.

In some embodiments, the probe or probe set comprises one or more barcode sequences or complements thereof. The barcode sequences may be positioned anywhere within the nucleic acid probe or probe set. If more than one barcode sequence is present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart. In some embodiments, one or more barcodes are indicative of a sequence in the target region of the target nucleic acid, such as a single nucleotide of interest (e.g., SNPs or point mutations), a dinucleotide sequence, or a short sequence of about 5 nucleotides in length.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

### III. Hybridization, Ligation, Extension, and Amplification

In some aspects, a method disclosed herein comprises one or more steps such as ligation, extension and/or amplification of the probe or probe set hybridized to the target nucleic acid. In some embodiments, the methods of the present application include the step of performing rolling circle amplification in the presence of a target nucleic acid of interest. In some embodiments, the hybridization and the ligation are carried out under the same reaction condition. For example, a ligase that performs the ligation is added prior to, during, and/or after the hybridization. In some embodiments, the ligase is present in and/or added to a reaction buffer for the hybridization. In some embodiments, the hybridization is performed using a buffer that is compatible with the ligation reaction. In some embodiments, the hybridization buffer is a ligase buffer supplemented with one or more RNase inhibitor(s). In some embodiments, the ligase buffer is further supplemented with formamide (e.g., 20% formamide), DMSO, or ethylene carbonate. In some embodiments, the ligase buffer is supplemented with a salt such as KCl (e.g., a ligase buffer supplemented with about 50 mM KCl). In some embodiments, supplementation of the ligase buffer with a salt and/or agents such as formamide, DMSO, or ethylene carbonate increases the stringency of hybridization. In some embodiments, the hybridization and ligation are carried out under the same reaction condition at a temperature of between about 30° C. and about 40° C. (e.g., about 37° C.). In some embodiments, the hybridization and ligation are carried out under the same reaction condition at a first temperature followed by a second temperature (e.g., a first incubation at 37° C. followed by a second incubation at a temperature between about 40° C. and about 45° C. In some embodiments, the method does not comprise washing the biological sample and/or changing a reaction buffer between the hybridization and the ligation. In some embodiments, the method does not comprise washing the biological sample and/or changing a reaction buffer between the contacting with the probe or probe set and the ligation.

In some embodiments of any of the methods disclosed herein, the method further comprises removing a complex comprising a probe or probe set and a decoy oligonucleotide (e.g., a decoy target) from the sample prior to a ligating step, prior to an amplification step, or prior to detecting the probe or probe set or a product thereof (e.g., a ligation or amplification product). In some embodiments, the method comprises removing complex comprising a ligatable probe or probe set and a decoy oligonucleotide from the sample prior to the ligating step. In some embodiments, the ligatable probe or probe set is a circularizable probe or probe set. In some embodiments, the method comprises removing a complex comprising a probe or probe set and a decoy oligonucleotide prior to rolling circle amplification.

In some aspects, provided herein is a method for analyzing a biological sample, comprising contacting the biological sample, a circularizable probe or probe set, and a decoy oligonucleotide with one another in any suitable order, wherein the biological sample comprises a target nucleic acid comprising a target region, the circularizable probe or probe set comprises a first hybridization region and a second hybridization region which, upon hybridization to the target region, are ligatable, and the decoy oligonucleotide comprises a decoy region capable of hybridizing to the first and/or second hybridization regions; allowing the circularizable probe or probe set and the target nucleic acid to hybridize at one or more locations in the biological sample, wherein the decoy oligonucleotide reduces hybridization between the first and/or second hybridization regions and an off-target region in the biological sample; circularizing the circularizable probe or probe set to generate a circular probe by ligating the first and second hybridization regions using the target region as template, with or without flap cleavage and with or without gap filling prior to ligation; generating a rolling circle amplification (RCA) product of the circular probe; and detecting a signal associated with the RCA product at the one or more locations, thereby detecting the target nucleic acid in the biological sample. In some embodiments, the circularizable probe or probe set is pre-hybridized to the decoy oligonucleotide. In some embodiments, the target region and/or the off-target region is pre-hybridized to the decoy oligonucleotide. In some embodiments, the first and/or hybridization regions displace the decoy region hybridized to the target region, thereby hybridizing the circularizable probe or probe set to the target nucleic acid. In some embodiments, the first and/or second hybridization regions do not displace the decoy region hybridized to the off-target region.

In some embodiments, the circularized probe is formed using ligation. In some embodiments, the circularized probe is formed using templated primer extension followed by ligation. In some embodiments, the circularized probe is formed by providing an insert between ends to be ligated. In some embodiments, the circularized probe is formed using a combination of any of the foregoing. In some embodiments, the ligation is a DNA-DNA templated ligation. In some embodiments, the ligation is an RNA-RNA templated ligation. In some embodiments, the ligation is a RNA-DNA templated ligation. In some embodiments, a splint is provided as a template for ligation. In some embodiments, the ligation is performed in the same reaction conditions as the hybridization reaction.

In some embodiments, the circularized probe is directly hybridized to the target nucleic acid. In some embodiments, the circularized probe is formed from a padlock probe. In some embodiments, the circularized probe is formed from a probe or probe set capable of DNA-templated ligation. See, e.g., U.S. Pat. No. 8,551,710, which is hereby incorporated by reference in its entirety. In some embodiments, the circularized probe is formed from a probe or probe set capable of RNA-templated ligation. Exemplary RNA-templated ligation probes and methods are described in US 2020/022424 which is incorporated herein by reference in its entirety. In some embodiments, the circularized probe is formed from a specific amplification of nucleic acids via intramolecular ligation (e.g., SNAIL) probe set. See, e.g., U.S. Pat. Pub. 20190055594, which is hereby incorporated by reference in its entirety. In some embodiments, the circularized probe is formed from a probe capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, the circularized probe is indirectly hybridized to the target nucleic acid. In some embodiments, the circularized probe is formed from a probe set capable of proximity ligation, for instance a proximity ligation in situ hybridization (PLISH) probe set. In some embodiments, a 3′ end and a 5′ end of the circularizable probe or probe set can be ligated using the target nucleic acid (e.g., RNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, the ligating step may comprise any ligation such as enzymatic ligation, chemical ligation, template dependent ligation, and/or template independent ligation. In any of the embodiments herein, the ligation can comprise using a ligase having an RNA-templated DNA ligase activity and/or an RNA-templated RNA ligase activity. In any of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments herein, the ligation can comprise using a PBCV-1 DNA ligase or variant or derivative thereof and/or a T4 RNA ligase 2 (T4 Rn12) or variant or derivative thereof.

In one aspect, provided herein is a method for analyzing a biological sample comprising contacting the sample with a ligatable probe or probe set comprising a split hybridization region complementary to a target region in a target nucleic acid, and a decoy oligonucleotide comprising a decoy region capable of hybridizing to the split hybridization region and/or the target region in the sample. In some embodiments, the decoy oligonucleotide reduces hybridization between the split hybridization region and an off-target region (e.g., by competing with the probe or probe set or with the off-target region for hybridization to the off-target region or the probe or probe set, respectively). In some embodiments, the ligation is carried out in the same reaction conditions as the hybridization. For example, ligation may be performed using a buffer that is also compatible with hybridization, with said buffer being added during the contacting step or during the hybridization step. In some embodiments, the ligase that performs the ligation is added prior to, during, and/or after the hybridization. In some embodiments, the ligase that performs the ligation is added prior to or during the hybridization. In some embodiments, the ligase is present in and/or added to a reaction buffer for the hybridization. In some embodiments, the method does not comprise washing the biological sample and/or changing a reaction buffer between hybridization and ligation. In some embodiments, the method does not comprise washing the biological sample and/or changing a reaction buffer between contacting the sample with the probe or probe set and performing the ligation.

In some embodiments, the method comprises removing molecules of the circularizable probe or probe set that are not bound to the target nucleic acid from the biological sample, molecules of the circularizable probe or probe set bound to a decoy oligonucleotide or having one or more mismatches with the target nucleic acid, and/or molecules of the decoy oligonucleotide bound to an off-target nucleic acid. For instance, one or more stringency washes can be used to remove circularizable probe molecules that are not bound to the target nucleic acid and/or bound to a decoy oligonucleotide.

Following formation of, e.g., the circularized probe or otherwise providing a circular probe, in some instances, an amplification primer is added. In other instances, the amplification primer is added with the probe or probe set. In some instances, the amplification primer may also be complementary to the target nucleic acid and the probe (e.g., a SNAIL probe). In some embodiments, a washing step is performed to remove any unbound probes, primers, etc. In some embodiments, the wash is a stringency wash. In some embodiments, the stringency is increased in the hybridization of the probe or probe set to the target nucleic acid, reducing or negating the need of performing a stringency wash.

In some embodiments, the probe or probe set is amplified in the sample. In some embodiments, the probe or probe set is a circular probe or circularizable probe or probe set, and the circular probe or a circularized probe generated from the circularizable probe or probe set is amplified in the sample. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof. In some embodiments, the amplification is performed at a temperature between or between about 20° C. and about 60° C. In some embodiments, the amplification is performed at a temperature between or between about 30° C. and about 40° C. The amplification product can be generated any suitable polymerase, including but not limited to Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

Upon addition of a DNA polymerase in the presence of appropriate dNTP precursors and other cofactors, the amplification primer is elongated by replication of multiple copies of the template to generate an amplification product (e.g., a concatemer of the template is generated). In any of the embodiments herein, the product can be immobilized in the biological sample. In any of the embodiments herein, the product can be crosslinked to one or more other molecules in the biological sample. This amplification product can be detected using, e.g., the secondary and higher order probes and detection oligonucleotides described herein. In some embodiments, the sequence of the amplicon or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. Suitable methods of detecting and/or analyzing probes or probe sets or products thereof are described in greater detail in Section IV. In some embodiments, the methods provided herein do not comprise performing rolling circle amplification. Any alternative methods of signal amplification such as those described in Section IV below may be used alternatively or in addition to rolling circle amplification.

### IV. Detection and Analysis

In some aspects, after formation of a hybridization complex comprising nucleic acid probes and/or probe sets described in Section II and any one or more optional further processing steps (e.g., ligation, extension, amplification or any combination thereof) as described in Section III, the method can comprise detection of the probe or probe set hybridized to the target nucleic acid or any products generated therefrom. In some embodiments, the decoy oligonucleotide (e.g., decoy target or decoy probe) reduces the detection of off-target signals in a detecting and/or imaging step.

In some embodiments, the method comprises imaging the sample to detect a signal associated with a probe hybridized to a target region in the sample. In some embodiments, the probe hybridized to the target region comprises one or more barcode sequences for detection and/or a means for signal amplification (such as an HCR initiator sequence). In some embodiments, the signal can be amplified in situ in the sample. In some embodiments, the signal amplification in situ comprises RCA of a probe that directly or indirectly binds to the ligated probe or probe set and/or the amplification product thereof; hybridization chain reaction (HCR) directly or indirectly on the probe or probe set and/or a product thereof (e.g., ligation and/or amplification product); linear oligonucleotide hybridization chain reaction (LO-HCR) directly or indirectly on the probe and/or a product thereof (e.g., ligation and/or amplification product); primer exchange reaction (PER) directly or indirectly on the probe and/or a product thereof (e.g., ligation and/or amplification product); assembly of branched structures directly or indirectly on the probe and/or a product thereof (e.g., ligation and/or amplification product); hybridization of a plurality of detectable probes directly or indirectly on the probe and/or a product thereof (e.g., ligation and/or amplification product), or any combination thereof.

In some embodiments, the method can comprise imaging the biological sample to detect a signal associated with a probe or probe set hybridized to a target region or product thereof (e.g., ligation and/or amplification product). In any one of the embodiments herein, a sequence of the probe or probe set, ligation product, rolling circle amplification product, or other generated product can be analyzed in situ in the biological sample. In any one of the embodiments herein, the imaging can comprise detecting a signal associated with a fluorescently labeled probe that directly or indirectly binds to a rolling circle amplification product of the circularized probe. In any one of the embodiments herein, the sequence of the probe or probe set, ligation product, rolling circle amplification product, extension product, or other generated product can be analyzed by sequential hybridization, sequencing by hybridization, sequencing by ligation, sequencing by synthesis, sequencing by binding, or a combination thereof.

In any one of the embodiments herein, a sequence associated with the target nucleic acid or the probe(s) can comprise one or more barcode sequences or complements thereof. In any one of the embodiments herein, the sequence of the rolling circle amplification product can comprise one or more barcode sequences or complements thereof. In any one of the embodiments herein, a ligated linear probe (e.g., generated from a first and second probe described herein) can comprise one or more barcode sequences or complements thereof. In some embodiments, a ligated linear probe can comprise an overhang region (e.g., a region that does not hybridize to the target nucleic acid) comprising one or more barcode sequences or complements thereof, which can be detected according to any of the methods described herein (optionally, wherein the detection comprises signal amplification). In some embodiments, a ligated linear probe can be released from the target nucleic acid (e.g., by rNase H digestion) prior to detecting a sequence of the ligated linear probe. In any one of the embodiments herein, the one or more barcode sequences can comprise a barcode sequence corresponding to the target nucleic acid. In any one of the embodiments herein, the one or more barcode sequences can comprise a barcode sequence corresponding to the sequence of interest, such as variant(s) of a single nucleotide of interest.

In some aspects, any of the probe(s) described herein can comprise one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or UMI). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the probes disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labelled probes (e.g., detection probes (e.g., detection oligos) or barcode probes). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and 20210164039, which are hereby incorporated by reference in their entirety.

In any one of the embodiments herein, the detecting step can comprise contacting the biological sample with one or more detectably-labeled probes that directly or indirectly hybridize to the rolling circle amplification product, and dehybridizing the one or more detectably-labeled probes from the rolling circle amplification product. In any one of the embodiments herein, the contacting and dehybridizing steps can be repeated with the one or more detectably-labeled probes and/or one or more other detectably-labeled probes that directly or indirectly hybridize to the rolling circle amplification product.

In any one of the embodiments herein, the detecting step can comprise contacting the biological sample with one or more intermediate probes that directly or indirectly hybridize to the probe or probe set and/or a product thereof (e.g., ligation and/or amplification product), wherein the one or more intermediate probes are detectable using one or more detectably-labeled probes. In any one of the embodiments herein, the detecting step can further comprise dehybridizing the one or more intermediate probes and/or the one or more detectably-labeled probes from the probe or probe set and/or a product thereof (e.g., ligation and/or amplification product). In any one of the embodiments herein, the contacting and dehybridizing steps can be repeated with the one or more intermediate probes, the one or more detectably-labeled probes, one or more other intermediate probes, and/or one or more other detectably-labeled probes.

In some embodiments, the detection may be spatial, e.g., in two or three dimensions. In some embodiments, the detection may be quantitative, e.g., the amount or concentration of a primary nucleic acid probe (and of a target nucleic acid) or a stem-loop structure may be determined. In some embodiments, the primary probes, secondary probes, higher order probes, and/or detectably labeled probes may comprise any one of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple primary probes (e.g., circularizable primary probes), and optionally multiple secondary probes hybridizing to the primary barcodes (or complementary sequences thereof) are all hybridized at once, followed by sequential secondary barcode detection and decoding of the signals. In some embodiments, detection of barcodes or subsequences of the barcode can occur in a cyclic manner.

In some embodiments, a method for analyzing a target region in a target nucleic acid is a multiplexed assay where multiple probes (e.g., circularizable probes) are used to detect multiple regions of interest simultaneously (e.g., variations at the same location of a target nucleic acid and/or SNPs in various locations). In some embodiments, one or more detections of one or more regions of interest may occur simultaneously. In some embodiments, one or more detections of one or more regions of interest may occur sequentially. In some embodiments, multiple circularizable probes of the same circularizable probe design are used to detect one or more regions of interest, using different barcodes associated with each target region. In some embodiments, multiple circularizable probes of different circularizable probe design are used to detect one or more regions of interest, using different barcodes (e.g., each barcode associated with a target nucleic acid or sequence thereof). In some embodiments, the one or more regions of interest are localized on the same molecule (e.g., RNA or DNA). In alternative embodiments, the one or more single nucleotides of interest are localized on different molecules.

In some aspects, the provided methods involve analyzing, e.g., detecting or determining, one or more sequences present in the polynucleotides (e.g., probe or probe set) and/or in a product or derivative thereof, such as in an amplified circularized probe. In some embodiments, the detection comprises providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a target region in a target nucleic acid. In other embodiments, the provided methods involve analyzing, e.g., detecting or determining, one or more sequences present in the polynucleotide probes (e.g., a barcode sequence or present in a probe or product thereof) or a cleaved stem-loop structure or a fragment thereof.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product generated using a method disclosed herein can be detected in with a method that comprises signal amplification.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US20220026433, WO 2020/102094, US20220128565, WO 2020/163397, and US20210222234, WO 2021/067475, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an initiator nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be referred to as the toehold region (or input domain). The first HCR monomers each further comprise a sequence which is complementary to a sequence in the exposed toehold region of the second HCR monomers. This sequence of complementarity in the first HCR monomers may be referred to as the interacting region (or output domain). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as metastable), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., US20200377926, which is herein incorporated by reference in its entirety), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any one of the embodiments herein, the target nucleic acid molecule and/or the analyte can be an RCA product.

In some embodiments, detection of nucleic acids sequences in situ includes detection of the probe or probe set and/or a product thereof (e.g., ligation and/or amplification product) with an assembly for branched signal amplification. In some embodiments, the assembly complex comprises an amplifier hybridized directly or indirectly (via one or more oligonucleotides) to a sequence of the probe or probe set and/or a product thereof (e.g., ligation and/or amplification product). In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. Nos. US20220064697 and US20200399689A1 and Xia et al., Multiplexed Detection of RNA using MERFISH and branched DNA amplification. Scientific Reports (2019), each of which is fully incorporated by reference herein.

In some embodiments, the probe or probe set and/or a product thereof (e.g., ligation and/or amplification product) can be detected with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes. See for example, U.S. Pat. Pub. No. US20190106733, which is incorporated herein by reference, for exemplary molecules and PER reaction components.

In some embodiments, the product or derivative of a first and second probe ligated together after hybridizing to the target nucleic acid can be analyzed by in situ sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing by synthesis.

In some embodiments, the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. A label or detectable label can be a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, yPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence can comprise background fluorescence (that can arise from a variety of sources, including aldehyde fixation, extracellular matrix components, red blood cells, lipofuscin, and the like), which is distinct from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as 125I, 35S, 14C, or 3H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. A fluorescent label can comprise a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Nucleotides having other fluorophores can also be synthesized (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,192,782. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any one of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequences can be analyzed in situ, e.g., by incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ analysis are described, for example, in Mitra et al., (2003) Anal. Biochem. 320, 55-65, and Lee et al., (2014) Science, 343(6177), 1360-1363; US 2016/0024555; US 2019/0194709; U.S. Pat. Nos. 10,138,509; 10,494,662; 10,179,932.

In some cases, sequencing can be performed after the analytes are released from the biological sample. In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, the method can comprise detecting the one or more barcode sequences in the probe or probe set or product thereof by contacting the biological sample with one or more detectably-labeled probes that directly or indirectly hybridize to the one or more barcode sequences, detecting signals associated with the one or more detectably-labeled probes, and dehybridizing the one or more detectably-labeled probes. In some embodiments, the contacting, detecting, and dehybridizing steps are repeated with the one or more detectably-labeled probes and/or one or more other detectably-labeled probes that directly or indirectly hybridize to the one or more barcode sequences. In some embodiments, the detectably labeled probes comprise a detectable label (e.g., are conjugated to a detectable label). In some embodiments, the detectably labeled probes are labeled with a sequence capable of hybridizing to a detection probe, wherein the detection probe comprises a detectable label (e.g., is conjugated to a detectable label). Methods of detecting and/or analyzing a sequence by sequential hybridization of probes have been described, for example, in U.S. Pat. Pub. 20210340618, the content of which is herein incorporated by reference in its entirety.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the probe or probe set or complements or products thereof are targeted by detectably labeled detection oligonucleotides, such as fluorescently labeled oligonucleotides. In some embodiments, one or more decoding schemes are used to decode the signals, such as fluorescence, for sequence determination. In any one of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

### V. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes and/or decoy oligonucleotides) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., one or more cycles as described in Section IV). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., any of the target analytes, labelling agents, or products described in Section III) in their naturally occurring place (e.g., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample (e.g., addition of fluids containing the probes or probe sets and/or decoy oligonucleotides). Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

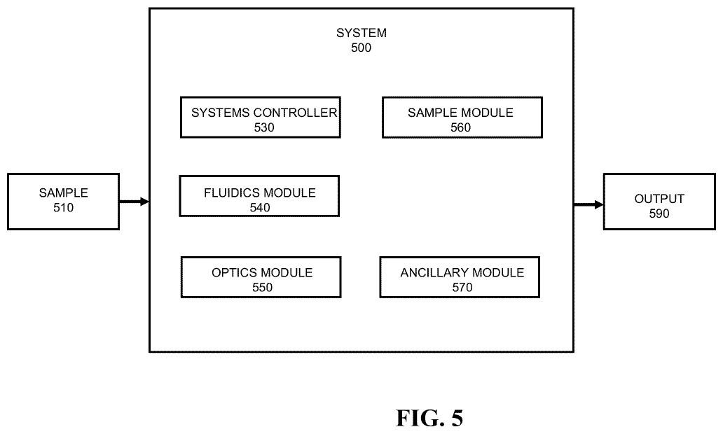

FIG. 5 shows an example workflow of analysis of a biological sample 510 (e.g., cell or tissue sample) using an opto-fluidic instrument 520, according to various embodiments. In various embodiments, the sample 510 can be a biological sample (e.g., a tissue) that includes molecules such as DNA, RNA, proteins, antibodies, etc. For example, the sample 510 can be a sectioned tissue that is treated to access the RNA thereof for labeling with probes described herein (e.g., in Section VIII). Ligation of a circularizable probe or probe set may generate a circular probe which can be enzymatically amplified and bound with detectably labeled probes, which can create bright signal that is convenient to image and has a high signal-to-noise ratio.

In various embodiments, the sample 510 may be placed in the opto-fluidic instrument 520 for analysis and detection of the molecules in the sample 410. In various embodiments, the opto-fluidic instrument 520 can be a system configured to facilitate the experimental conditions conducive for the detection of the target molecules. For example, the opto-fluidic instrument 520 can include a fluidics module 540, an optics module 550, a sample module 560, and an ancillary module 570, and these modules may be operated by a system controller 530 to create the experimental conditions for the probing of the molecules in the sample 510 by selected probes (e.g., circularizable DNA probes), as well as to facilitate the imaging of the probed sample (e.g., by an imaging system of the optics module 550). In various embodiments, the various modules of the opto-fluidic instrument 520 may be separate components in communication with each other, or at least some of them may be integrated together.

In various embodiments, the sample module 560 may be configured to receive the sample 510 into the opto-fluidic instrument 520. For instance, the sample module 560 may include a sample interface module (SIM) that is configured to receive a sample device (e.g., cassette) onto which the sample 510 can be deposited. That is, the sample 510 may be placed in the opto-fluidic instrument 520 by depositing the sample 510 (e.g., the sectioned tissue) on a sample device that is then inserted into the SIM of the sample module 560. In some instances, the sample module 560 may also include an X-Y stage onto which the SIM is mounted. The X-Y stage may be configured to move the SIM mounted thereon (e.g., and as such the sample device containing the sample 510 inserted therein) in perpendicular directions along the two-dimensional (2D) plane of the opto-fluidic instrument 520.

The experimental conditions that are conducive for the detection of the molecules in the sample 510 may depend on the target molecule detection technique that is employed by the opto-fluidic instrument 520. For example, in various embodiments, the opto-fluidic instrument 520 can be a system that is configured to detect molecules in the sample 510 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 510 using reagents such as washing/stripping reagents, hybridizing reagents, etc., and such reagents may be provided by the fluidics module 540.

In various embodiments, the fluidics module 540 may include one or more components that may be used for storing the reagents, as well as for transporting said reagents to and from the sample device containing the sample 510. For example, the fluidics module 540 may include reservoirs configured to store the reagents, as well as a waste container configured for collecting the reagents (e.g., and other waste) after use by the opto-fluidic instrument 520 to analyze and detect the molecules of the sample 510. Further, the fluidics module 540 may also include pumps, tubes, pipettes, etc., that are configured to facilitate the transport of the reagent to the sample device (e.g., and as such the sample 510). For instance, the fluidics module 540 may include pumps (“reagent pumps”) that are configured to pump washing/stripping reagents to the sample device for use in washing/stripping the sample 510 (e.g., as well as other washing functions such as washing an objective lens of the imaging system of the optics module 550).

In various embodiments, the ancillary module 570 can be a cooling system of the opto-fluidic instrument 520, and the cooling system may include a network of coolant-carrying tubes that are configured to transport coolants to various modules of the opto-fluidic instrument 520 for regulating the temperatures thereof. In such cases, the fluidics module 540 may include coolant reservoirs for storing the coolants and pumps (e.g., “coolant pumps”) for generating a pressure differential, thereby forcing the coolants to flow from the reservoirs to the various modules of the opto-fluidic instrument 520 via the coolant-carrying tubes. In some instances, the fluidics module 540 may include returning coolant reservoirs that may be configured to receive and store returning coolants, e.g., heated coolants flowing back into the returning coolant reservoirs after absorbing heat discharged by the various modules of the opto-fluidic instrument 520. In such cases, the fluidics module 540 may also include cooling fans that are configured to force air (e.g., cool and/or ambient air) into the returning coolant reservoirs to cool the heated coolants stored therein. In some instance, the fluidics module 540 may also include cooling fans that are configured to force air directly into a component of the opto-fluidic instrument 520 so as to cool said component. For example, the fluidics module 540 may include cooling fans that are configured to direct cool or ambient air into the system controller 530 to cool the same.

As discussed above, the opto-fluidic instrument 520 may include an optics module 550 which include the various optical components of the opto-fluidic instrument 520, such as but not limited to a camera, an illumination module (e.g., LEDs), an objective lens, and/or the like. The optics module 550 may include a fluorescence imaging system that is configured to image the fluorescence emitted by the probes (e.g., oligonucleotides) in the sample 510 after the probes are excited by light from the illumination module of the optics module 550.

In some instances, the optics module 550 may also include an optical frame onto which the camera, the illumination module, and/or the X-Y stage of the sample module 560 may be mounted.

In various embodiments, the system controller 530 may be configured to control the operations of the opto-fluidic instrument 520 (e.g., and the operations of one or more modules thereof). In some instances, the system controller 530 may take various forms, including a processor, a single computer (or computer system), or multiple computers in communication with each other. In various embodiments, the system controller 530 may be communicatively coupled with data storage, set of input devices, display system, or a combination thereof. In some cases, some or all of these components may be considered to be part of or otherwise integrated with the system controller 530, may be separate components in communication with each other, or may be integrated together. In other examples, the system controller 530 can be, or may be in communication with, a cloud computing platform.

In various embodiments, the opto-fluidic instrument 520 may analyze the sample 510 and may generate the output 590 that includes indications of the presence of the target molecules in the sample 510. For instance, with respect to the example embodiment discussed above where the opto-fluidic instrument 520 employs a hybridization technique for detecting molecules, the opto-fluidic instrument 520 may cause the sample 510 to undergo successive rounds of detectably labeled probe hybridization (e.g., using two or more sets of fluorescent probes, where each set of fluorescent probes is excited by a different color channel) and be imaged to detect target molecules in the probed sample 510. In such cases, the output 590 may include optical signatures (e.g., a codeword) specific to each gene, which allow the identification of the target molecules.

### VI. Compositions and Kits

Also provided herein are kits, for example comprising one or more polynucleotides (e.g., any of the probes or probe sets and/or decoy oligonucleotides described in Section III) and reagents for performing the methods disclosed herein. In some embodiments, the kit comprises one or more reagents required for one or more hybridization, ligation, amplification, detection, and/or sample preparation steps as described herein. In some embodiments, the kit further comprises a target nucleic acid (e.g., a target nucleic acid in a labeling agent described in Section VII). In some embodiments, any or all of the polynucleotides (e.g., probe, probe set, and/or decoy oligonucleotide) are DNA molecules. In some embodiments, the decoy oligonucleotide is a decoy target, such as any of the decoy targets described in Section II.A.(i). In some embodiments, the decoy oligonucleotide is a decoy probe, such as any of the decoy probes described in Section II.A.(ii). In some embodiments, the target nucleic acid is a messenger RNA molecule. In some embodiments, the kit further comprises one or more ligases, for instance for forming a ligated probe from a probe or probe set (e.g., a ligated circular probe or a ligated linear probe). In some embodiments, the kit further comprises a polymerase, for instance for performing amplification circular or circularized probe or probe set, e.g., using any of the methods described in Section III. In some embodiments, the polymerase is Phi29. In some embodiments, the kit further comprises a primer for amplification of the probe or probe set. In some embodiments, the kit further comprises one or more detection reagents such as those disclosed in Section IV.

In some embodiments, provided herein is a kit comprising a probe or probe set, a first decoy oligonucleotide, and a second decoy oligonucleotide, wherein the first decoy oligonucleotide comprises a first decoy region having a first percent complementarity to a hybridization region of the probe or probe set or to a target region complementary to the hybridization region, and the second decoy oligonucleotide comprises a second decoy region having a second percent complementarity to the hybridization region of the probe or probe set or to the target region. In some embodiments, the first decoy oligonucleotide is provided at a first concentration and the second decoy oligonucleotide is provided at a second concentration. In some embodiments, the first percent complementarity is higher than the second percent complementarity. In some embodiments, the first concentration is lower than the second concentration.

In some embodiments, provided herein is a kit for analyzing a biological sample comprising a plurality of probes or probe sets, wherein each probe or probe set is designed to hybridize to a target region in a target nucleic acid, wherein one or more of the target regions comprise a sub-sequence that occurs in off-target molecules present in a genome or transcriptome of the biological sample, and wherein the kit comprises one or more decoy oligonucleotides complementary to the sub-sequence of the target region.

In some embodiments, provided herein is a complex comprising a decoy oligonucleotide hybridized to a probe or probe set. In some embodiments, provided herein is a kit comprising a probe or probe set comprising a hybridization region complementary to the target region, and a decoy oligonucleotide, wherein the decoy oligonucleotide comprises a decoy region having at least 80%, at least 85%, at least 90%, or at least 95% sequence identity to the target region. In some embodiments, the decoy region is hybridized to the hybridization region in the probe or probe set (e.g., the probe or probe set and the decoy oligonucleotide are provided as a complex.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect a target region in target nucleic acids.

In some embodiments, the target region comprises more than one nucleotide of interest. In some embodiments, the target region comprises an alternatively spliced region, a deletion, and/or a frameshift. In some embodiments, the target region comprises a single nucleotide of interest. In some embodiments, the single nucleotide of interest is a single-nucleotide polymorphism (SNP). In some embodiments, the single nucleotide of interest is a single-nucleotide variant (SNV). In some embodiments, the single nucleotide of interest is a single-nucleotide substitution. In some embodiments, the single nucleotide of interest is a point mutation. In some embodiments, the single nucleotide of interest is a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Samples and Analytes

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a predisposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, a cell pellet, a cell block, a biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Sample Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. In some embodiments, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., one or more probes or probe sets and one or more decoy oligonucleotides. In some embodiments, one or more post-fixing step is performed after a hybridization complex comprising a probe and a target is formed in a sample.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences. A post-fixing step may be performed using any suitable fixation reagent disclosed herein, for example, 3% (w/v) paraformaldehyde in DEPC-PBS.

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (ii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(iii) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (iv) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (v) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, dNase and rNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (vi) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some embodiments, the target region is in or is associated with an analyte. In some embodiments, one or more off-target regions are present in the biological sample. In some embodiments, the one or more off-target regions are present in a different molecule in the sample (e.g., the target analyte is an endogenous RNA and the off-target region is present in an off-target DNA or RNA molecule in the sample). In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific subcellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### IX. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample). A barcode can be part of an analyte, or independent of an analyte. A barcode can be attached to an analyte. A barcode can be in a probe or probe set. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be associated with an analyte by its inclusion in a probe or probe set that hybridizes to an analyte. A barcode can be added to, for example, a fragment of a deoxyribonucleic acid (DNA) or ribonucleic acid (RNA) sample before or during sequencing of the sample.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

(viii) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a polynucleotide disclosed herein (e.g., a probe, probe set, or decoy oligonucleotide). For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following example is included for illustrative purposes only and is not intended to limit the scope of the present disclosure.

### Example 1: Use of Decoy Oligonucleotides to Reduce Off-Target Hybridization In Situ in a Biological Sample

This example describes the use of an in situ hybridization probe and a decoy oligonucleotide for detection of a target region in a target nucleic acid (e.g., an mRNA) in situ in a biological sample.

A tissue sample is obtained and cryosectioned onto a glass slide for processing. The tissue is fixed by incubating in 3.7% paraformaldehyde (PFA). One or more washes is performed and the tissue is then permeabilized. To prepare for probe hybridization, a wash buffer is added to the tissue section.

A probe and decoy oligonucleotide mixture is incubated with the tissue section sample and hybridization buffer for hybridization of the probes to target nucleic acid (e.g., mRNAs) in the sample. The probe set mixture comprises a probe that hybridizes to a target region in a target nucleic acid as depicted in FIGS. 1-2, wherein the probe comprises at least one overhang region that does not hybridize to the target nucleic acid. The decoy oligonucleotide can be any of the decoy oligonucleotides described in Section II.A (e.g., a decoy target or a decoy probe) and depicted in FIGS. 1-4D.

Optionally, the sample can be washed to remove unbound probes. The hybridized probe can then be detected using any of the signal amplification techniques described in Section IV (e.g., hybridization of a circular or circularizable probe to the overhang region and subsequent rolling circle amplification, hybridization chain reaction (HCR) directly or indirectly on the overhang region of the probe, linear oligonucleotide hybridization chain reaction (LO-HCR) directly or indirectly on the overhang region of the probe, primer exchange reaction (PER) directly or indirectly on the overhang region of the probe; assembly of branched structures directly or indirectly on the overhang region of the probe; hybridization of a plurality of detectable probes directly or indirectly on the overhang region of the probe, or any combination thereof.

In the absence of a decoy oligonucleotide, the probe hybridizes to an off-target region, resulting in a false positive signal as shown in the top panels of FIGS. 1-2. In the presence of the decoy oligonucleotide, the probe does not hybridize to the off-target region, resulting in the absence of a false positive signal as shown in the bottom panels of FIGS. 1-2.

### Example 2: Simultaneous Hybridization and Ligation of Probes in the Presence of a Decoy Oligonucleotide

This example describes the use of a ligatable probe or probe set (e.g., a circularizable probe or a ligatable first and second probe) and a decoy oligonucleotide, wherein hybridization and ligation are performed simultaneously (or are not separated by a wash step).

A tissue sample is prepared as described in Example 1 above. A probe mixture comprising a (i) ligatable probe set comprising a first hybridization region and a second hybridization region and (ii) a decoy oligonucleotide in a buffer is incubated with the tissue section sample. The buffer can comprise an RNase inhibitor. A ligase can be included in the same buffer (e.g., PBCV-1 ligase) for ligation of the first hybridization region and second hybridization region. Hybridization and ligation can be performed at 37° C. for at least 30 min (e.g., at 37° C. for 30 min and then moved to 45° C. for 1.5 h). Alternatively, the ligase can be contacted with the sample after hybridizing the probes, omitting a wash step before contacting the sample with the ligase.

The decoy oligonucleotide can be any of the decoy oligonucleotides described in Section II.A (e.g., a decoy target or a decoy probe) and depicted in FIGS. 1-4D. In an example, the sample includes an off-target nucleic acid comprising an off-target region that has significant sequence homology to the target region (e.g., at least 80% or at least 90% sequence identity to the target region).

The first hybridization region and the second hybridization region can hybridize to adjacent portions of a target region in a target nucleic acid, such as an mRNA molecule, such that a first and second ligatable end are juxtaposed for ligation. The ligatable probe can be any ligatable probe described in Section II.B (e.g., a circularizable probe, or a first and second probe).

After incubating the sample to allow hybridization and ligation of the ligatable probe or probe set, one or more stringent washes can be performed. In some examples, the sample is then contacted with a polymerase (e.g., Phi29) to perform rolling circle amplification of the probe (in the case of a circularizable probe or probe set).

The probe or probe set, or an amplification product (e.g., RCA product) thereof is detected according to any of the methods described in Section IV. For example, a barcode sequence in the probe or probe set or amplification product thereof can be detected by sequential hybridization of probes associated with detectable labels. In an example, the decoy oligonucleotide reduces detection of an off-target signal in situ at locations in the tissue sample compared to the amount of off-target signal detected in the absence of the decoy oligonucleotide.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
a) contacting the biological sample with a probe or probe set and a decoy oligonucleotide in any suitable order, wherein:
the biological sample comprises a target nucleic acid comprising a target region,
the probe or probe set comprises a hybridization region, and
the decoy oligonucleotide comprises a decoy region capable of hybridizing to the hybridization region or the target region;
b) allowing the probe or probe set and the target nucleic acid to hybridize at one or more locations in the biological sample, wherein the decoy oligonucleotide reduces hybridization between the hybridization region and an off-target region in the biological sample; and
c) detecting a signal associated with the probe or probe set or a product thereof at the one or more locations in the biological sample, thereby detecting the target nucleic acid in the biological sample.

2. The method of claim 1, wherein the decoy region has less than 98% sequence identity to the hybridization region of the probe or probe set.

3. The method of claim 2, wherein the hybridization region of the probe or probe set has at least 99% sequence complementarity to the target region.

4. The method of any of claims 1-3, wherein the decoy region has a lower sequence complementarity to the target region compared to the sequence complementarity of the hybridization region to the target region.

5. The method of any of claims 1-4, wherein the decoy region has less than 98% sequence complementarity to the target region.

6. The method of claim 5, wherein the decoy region has less than 95% sequence complementarity to the target region.

7. The method of claim 5 or 6, wherein the decoy region has between about 80% and about 95% sequence complementarity to the target region.

8. The method of any of claims 1-4, wherein the hybridization region has a higher sequence complementarity to the target region compared to the sequence complementarity of the hybridization region to the decoy region.

9. The method of claim 8, wherein the hybridization region has at least 95% sequence complementarity to the decoy region.

10. The method of claim 8 or 9, wherein the hybridization region has at least 99% sequence complementarity to the decoy region.

11. The method of any of claims 1-10, wherein the decoy oligonucleotide is no more than about 10, no more than about 15, no more than about 20, no more than about 25, no more than about 30, no more than about 35, no more than about 40, no more than about 45, no more than about 50, no more than about 60, no more than about 70, no more than about 80, no more than about 90, or no more than about 100 nucleotides in length.

12. The method of any of claims 1-11, wherein the decoy oligonucleotide is detectably labeled or not detectably labeled.

13. The method of any of claims 1-12, wherein upon hybridization to the off-target region or the hybridization region, the decoy oligonucleotide does not comprise a region capable of directly or indirectly binding to a detectably labeled probe.

14. The method of any of claims 1-13, wherein upon hybridization to the off-target region or the hybridization region, the decoy oligonucleotide is not ligatable with itself, within the probe set, or with another oligonucleotide.

15. The method of any of claims 1-14, wherein upon hybridization to the off-target region or the hybridization region, the decoy oligonucleotide is not detectable by detectable probes configured to detect the probe or probe set or product thereof.

16. The method of any of claims 1-15, wherein upon hybridization to the off-target region or the hybridization region, the decoy oligonucleotide is not capable of generating a product that is detectable by detectable probes configured to detect the probe or probe set or product thereof.

17. The method of any of claims 1-16, wherein the product is a rolling circle amplification (RCA) product.

18. The method of any of claims 1-17, wherein the probe or probe set is selected from the group consisting of: a probe comprising a 3′ or 5′ overhang upon hybridization to the target nucleic acid, optionally wherein the 3′ or 5′ overhang comprises one or more detectable labels and/or barcode sequences; a probe comprising a 3′ overhang and a 5′ overhang upon hybridization to the target nucleic acid, optionally wherein the 3′ overhang and the 5′ overhang each independently comprises one or more detectable labels and/or barcode sequences; a circular probe; a circularizable probe or probe set; a probe or probe set comprising a split hybridization region configured to hybridize to a splint, optionally wherein the split hybridization region comprises one or more barcode sequences; and a combination thereof.

19. The method of any of claims 1-18, wherein the probe or probe set is not detectably labeled.

20. The method of any of claims 1-19, wherein the probe or probe set further comprises a region capable of directly or indirectly binding to a detectably labeled probe.

21. The method of any of claims 1-20, wherein upon hybridization to the target region, the probe or probe set is ligatable with itself, within the probe set, or with another oligonucleotide.

22. The method of claim 21, wherein the probe or probe set is ligatable using the target region as template, with or without flap cleavage and with or without gap filling prior to ligation.

23. The method of any of claims 1-22, wherein upon hybridization to the target region, the probe or probe set is capable of generating a product.

24. The method of claim 23, wherein the product of the probe or probe set is a rolling circle amplification (RCA) product generated in situ in the biological sample.

25. The method of any of claims 1-24, wherein the method comprises prior to the detecting in c), a step of removing a complex comprising the probe or probe set hybridized to the decoy oligonucleotide from the biological sample.

26. A method for analyzing a biological sample, comprising:
a) contacting the biological sample, a circularizable probe or probe set, and a decoy oligonucleotide with one another in any suitable order, wherein:
the biological sample comprises a target nucleic acid comprising a target region,
the circularizable probe or probe set comprises a first hybridization region and a second hybridization region which, upon hybridization to the target region, are ligatable, and
the decoy oligonucleotide comprises a decoy region capable of hybridizing to the first and/or second hybridization regions;
b) allowing the circularizable probe or probe set and the target nucleic acid to hybridize at one or more locations in the biological sample, wherein the decoy oligonucleotide reduces hybridization between the first and/or second hybridization regions and an off-target region in the biological sample;
c) circularizing the circularizable probe or probe set to generate a circular probe by ligating the first and second hybridization regions using the target region as template, with or without flap cleavage and with or without gap filling prior to ligation;
d) generating a rolling circle amplification (RCA) product of the circular probe; and
e) detecting a signal associated with the RCA product at the one or more locations, thereby detecting the target nucleic acid in the biological sample.

27. The method of claim 26, wherein the circularizable probe or probe set is pre-hybridized to the decoy oligonucleotide.

28. The method of claim 27, wherein the target region displaces the decoy region hybridized to the circularizable probe or probe set, thereby hybridizing the circularizable probe or probe set to the target nucleic acid.

29. The method of any of claims 26-28, wherein the hybridization in step b) and the ligation in step c) are carried out under the same reaction condition, optionally wherein a ligase that performs the ligation is added prior to, during, and/or after the hybridization in step b).

30. The method of claim 29, wherein the ligase is present in and/or added to a reaction buffer for the hybridization in step b).

31. The method of any of claims 26-30, which does not comprise washing the biological sample and/or changing a reaction buffer between the hybridization in step b) and the ligation in step c).

32. The method of any of claims 26-31, which does not comprise washing the biological sample and/or changing a reaction buffer between the contacting in step a) and the ligation in step c).

33. The method of any of claims 26-32, which comprises prior to the circularizing in step c), a step of removing a complex comprising the circularizable probe or probe set hybridized to the decoy oligonucleotide from the biological sample.

34. The method of any of claims 26-32, wherein a complex comprising the circularizable probe or probe set hybridized to the decoy oligonucleotide is not removed from the biological sample prior to the circularizing in step c).

35. The method of claim 34, wherein in the complex, the decoy oligonucleotide comprises one or more mismatches with the circularizable probe or probe set at or near a ligation junction.

36. The method of claim 35, wherein in step c), a circular probe of the circularizable probe or probe set hybridized to the decoy oligonucleotide is not generated.

37. The method of any of claims 26-36, wherein the decoy oligonucleotide is not capable of being extended by a polymerase, optionally wherein the decoy oligonucleotide comprises an irreversible terminating group, optionally wherein the decoy oligonucleotide comprises a 3′ dideoxynucleotide.

38. A method for analyzing a biological sample, comprising:
a) contacting the biological sample, a circularizable probe or probe set, and a decoy oligonucleotide with one another in any suitable order, wherein:
the biological sample comprises a target nucleic acid comprising a target region,
the circularizable probe or probe set comprises a first hybridization region and a second hybridization region which, upon hybridization to the target region, are ligatable, and
the decoy oligonucleotide comprises a decoy region capable of hybridizing to the target region;
b) allowing the circularizable probe or probe set and the target nucleic acid to hybridize at one or more locations in the biological sample, wherein the decoy oligonucleotide reduces hybridization between the first and/or second hybridization regions and an off-target region in the biological sample;
c) circularizing the circularizable probe or probe set to generate a circular probe by ligating the first and second hybridization regions using the target region as template, with or without flap cleavage and with or without gap filling prior to ligation;
d) generating a rolling circle amplification (RCA) product of the circular probe; and
e) detecting a signal associated with the RCA product at the one or more locations, thereby detecting the target nucleic acid in the biological sample.

39. The method of claim 38, wherein the target region and/or the off-target region is pre-hybridized to the decoy oligonucleotide.

40. The method of claim 39, wherein the first and/or hybridization regions displace the decoy region hybridized to the target region, thereby hybridizing the circularizable probe or probe set to the target nucleic acid, and/or wherein the first and/or hybridization regions do not displace the decoy region hybridized to the off-target region.

41. The method of any of claims 38-40, wherein the hybridization in step b) and the ligation in step c) are carried out under the same reaction condition, wherein a ligase that performs the ligation is added prior to, during, and/or after the hybridization in step b).

42. The method of claim 41, wherein the ligase is present in and/or added to a reaction buffer for the hybridization in step b).

43. The method of any of claims 38-42, which does not comprise washing the biological sample and/or changing a reaction buffer between the hybridization in step b) and the ligation in step c).

44. The method of any of claims 38-43, which does not comprise washing the biological sample and/or changing a reaction buffer between the contacting in step a) and the ligation in step c).

45. The method of any of claims 38-44, wherein a first decoy oligonucleotide hybridized to the target region is removed from the biological sample prior to the circularizing in step c), and/or wherein a second decoy oligonucleotide hybridized to the off-target region is not removed from the biological sample prior to the circularizing in step c).

46. The method of claim 45, wherein the decoy oligonucleotide is circularizable, and the decoy oligonucleotide comprises one or more mismatches with the target region at or near a ligation junction.

47. The method of claim 45 or 46, wherein the decoy oligonucleotide does not comprise a mismatch with the off-target region at or near a ligation junction.

48. The method of any one of claims 45-46, wherein in the first complex and/or the second complex, the decoy oligonucleotide comprises a non-ligatable 3′ end and/or non-ligatable 5′ end.

49. The method of any one of claims 47-48, wherein in step c), a circular probe is not generated of the decoy oligonucleotide hybridized to the target region or the off-target region.

50. The method of any one of claims 1-49, wherein the decoy oligonucleotide lacks a phosphate group at the 5′ end.

51. The method of any one of claims 1-50, wherein the decoy oligonucleotide comprises one or more modifications that reduce its ability to be used as a template for amplification.

52. The method of any one of claims 1-4 or 8-37, wherein complementarity between the decoy oligonucleotide and the hybridization region in the probe or probe set is lower than complementarity between the hybridization region and the target region of the target nucleic acid.

53. The method of any one of claims 1-4, 8-37, or 52, wherein the decoy oligonucleotide has between about 80% and about 95% complementarity to the hybridization region in the probe or probe set.

54. The method of any one of claims 1-4, 8-37, or 52-53 wherein the probe or probe set and the decoy oligonucleotide are provided as the first complex.

55. The method of any one of claims 1-54, wherein the probe or probe set and the decoy oligonucleotide are provided separately.

56. The method of any one of claims 1-55, wherein the probe or probe set and the decoy oligonucleotide are provided at a ratio of 1:1.

57. The method any one of claims 1-56, wherein the probe or probe set and the decoy oligonucleotide are provided at a ratio higher than 1:1.

58. The method of any one of claims 55-57, wherein the method further comprises allowing hybridization of the probe or probe set and the decoy oligonucleotide to form the first complex.

59. The method of any one of claims 55-57, wherein the decoy oligonucleotide is no more than 10, no more than 15, no more than 20, no more than 25, or no more than 30 nucleotides in length.

60. The method of any of claims 1-59, wherein the method further comprises removing the decoy probe hybridized to the first and/or second hybridization region or the target region prior to ligating the probe or probe set hybridized to the target nucleic acid.

61. The method of claim 60, wherein the removing step comprises one or more stringency washes.

62. The method of any one of claims 1-61, wherein the target nucleic acid is an mRNA.

63. The method of any one of claims 1-61, wherein the target nucleic acid is a noncoding RNA.

64. The method of any one of claims 1-63, wherein the target region comprises a single nucleotide of interest, an alternatively spliced region, a deletion, and/or a frameshift.

65. The method of claim 64, wherein the single nucleotide of interest is selected from the group consisting of a single-nucleotide polymorphism (SNP), a single-nucleotide variant (SNV), a single-nucleotide substitution, a point mutation, or a single-nucleotide insertion.

66. The method of claim 65, wherein the single nucleotide of interest is a SNP.

67. The method of claim 65, wherein the single nucleotide of interest is a point mutation.

68. The method of any of claims 1-67, wherein the target biological sample is a tissue sample, and the target region is analyzed in situ at a location in the tissue sample.

69. The method of claim 68, wherein the tissue sample is an intact tissue sample or a non-homogenized tissue sample.

70. The method of claim 68 or claim 69, wherein the target nucleic acid is in a cell in the tissue sample.

71. The method of claim 70, further comprising permeabilizing the cell before, during, or after the contacting step.

72. The method of any of claims 68-71, wherein the tissue sample is a tissue section.

73. The method of any of claims 68-72, wherein the tissue sample is a fixed tissue sample, a frozen tissue sample, or a fresh tissue sample.

74. The method of any of claims 26-73, wherein the ligating is enzymatic ligation or chemical ligation.

75. The method of any one of claims 26-74, wherein the ligating is performed using a ligase selected from the group consisting of a T4 RNA ligase 1, a T4 RNA ligase 2 or a PBCV-1 DNA ligase.

76. The method of any one of claims 26-75, wherein ligating the probe or probe set results in a circularized probe.

77. The method of any of claims 26-76, wherein detecting the ligated probe or probe set comprises generating an amplification product in situ, and detecting the amplification product.

78. The method of claim 77, wherein detecting the probe or probe set, the ligated probe or probe set, and/or the amplification product comprises determining a sequence of the probe or probe set, the ligated probe or probe set, or the amplification product.

79. The method of claim 78, wherein the sequencing comprises sequencing by hybridization, sequencing by ligation, and/or fluorescent in situ sequencing.

80. The method of claim 78 or claim 79, wherein a sequence in the amplification product indicative of the target region is determined.

81. The method of claim 78, wherein the in situ hybridization comprises sequential fluorescent in situ hybridization.

82. The method of any one of claims 1-81, wherein detecting of the probe or probe set, the ligated probe or probe set, and/or the amplification product comprises labeling the ligated probe or probe set and/or the amplification product with a fluorophore, an isotope, a mass tag, or a combination thereof.

83. The method of any one of claims 15-82, wherein the amplification product is generated using a linear rolling circle amplification (RCA), a branched RCA, a dendritic RCA, or any combination thereof.

84. The method of claim 83, wherein the amplification product is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

85. The method of any of claims 1-84, wherein the probe or probe set, the ligated probe or probe set and/or the amplification product thereof are immobilized in the biological sample and/or crosslinked to one or more other molecules in the biological sample.

86. The method of any of claims 1-85, wherein the method comprises imaging the sample to detect the probe or probe set, the ligated probe or probe set, and/or the amplification product thereof.

87. The method of claim 86, wherein the imaging comprises detecting a signal associated the probe or probe set, the ligated probe or probe set, and/or the amplification product thereof.

88. The method of claim 87, wherein the signal is amplified in situ in the biological sample.

89. The method of claim 88, wherein the signal amplification in situ comprises RCA of a probe that directly or indirectly binds to the probe or probe set; hybridization chain reaction (HCR) directly or indirectly on the probe or probe set and/or the product thereof; linear oligonucleotide hybridization chain reaction (LO-HCR) directly or indirectly on the probe or probe set and/or the product thereof; primer exchange reaction (PER) directly or indirectly on the probe or probe set and/or the product thereof; assembly of branched structures directly or indirectly on the probe or probe set and/or the product thereof, hybridization of a plurality of detectable probes directly or indirectly on the probe or probe set and/or the product thereof, or any combination thereof.

90. The method of any one of claims 1-89 wherein the probe or probe set comprise one or more barcode sequences.

91. The method of any of claims 1-90, wherein the probe or probe set comprise one or more barcode sequences that identifies a nucleic acid sequence.

92. The method of claim 91, wherein the one or more barcode sequences identify the target region.

93. The method of any one of claims 90-92, wherein the one or more barcode sequences are between about 8 and about 16 nucleotides in length.

94. The method of claim 93, wherein the one or more barcode sequences are between about 8 and about 10 nucleotides in length.

95. The method of any one of claims 90-94, wherein the method comprises detecting the one or more barcode sequences by:
contacting the biological sample with one or more detectably-labeled probes that directly or indirectly hybridize to the one or more barcode sequences,
detecting signals associated with the one or more detectably-labeled probes, and
dehybridizing the one or more detectably-labeled probes,
optionally wherein the contacting, detecting, and dehybridizing steps are repeated with the one or more detectably-labeled probes and/or one or more other detectably-labeled probes that directly or indirectly hybridize to the one or more barcode sequences.

96. A method for analyzing a biological sample, comprising:
a) contacting the biological sample, with a complex comprising a circularizable probe and a decoy oligonucleotide, wherein:
the biological sample comprises a target nucleic acid comprising a target region,
the circularizable probe comprises a first hybridization region and a second hybridization region which, upon hybridization to the target region, are ligatable, and
the decoy oligonucleotide comprises a decoy region capable of hybridizing to the first and/or second hybridization regions, wherein the complementarity between the decoy region and the first and/or second hybridization region is lower than the complementarity between the target region and the first and/or second hybridization region, but the complementarity between the decoy region and the first and/or second hybridization region is higher than the complementarity between an off-target region and the first and/or second hybridization region;
b) allowing the circularizable probe to hybridize to the target nucleic acid at one or more locations in the biological sample, thereby displacing the decoy oligonucleotide;
c) circularizing the circularizable probe to generate a circular probe by ligating the first and second hybridization regions using the target region as template, wherein the ligating is performed under the same reaction conditions as the hybridizing in step b);
d) generating a rolling circle amplification (RCA) product of the circular probe; and
e) detecting a signal associated with the RCA product at the one or more locations, thereby detecting the target nucleic acid in the biological sample.

97. A kit for analyzing a biological sample, comprising:
(i) a probe or probe set comprising a hybridization region complementary to a target region in a target nucleic acid,
(ii) a first decoy oligonucleotide, wherein the first decoy oligonucleotide comprises a first decoy region having a first percent complementarity to a hybridization region of the probe or probe set or to the target region, and
(iii) a second decoy oligonucleotide, wherein the second decoy oligonucleotide comprises a second decoy region having a second percent complementarity to the hybridization region of the probe or probe set or to the target region.

98. The kit of claim 97, wherein the first percent complementarity is higher than the second percent complementarity.

99. The kit of claim 97 or 98, wherein the first decoy oligonucleotide is at a first concentration and the second decoy oligonucleotide is at a second concentration.

100. The kit of claim 99, wherein the first concentration is lower than the second concentration.
